SEQUENCE LISTING (31 sequences)

SEQ ID NO: 1 - protein, 781 aa, Vibrio cholerae
MRFKNVKKTALMLAMFGMATSSNAALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDVIFRGPDRIPSIVASSVTPGVVTAFAEKRVGGGDPGALSNTNDIITRTSRDGGITWDTELNLTEQINVSDEFDFSDPRPIYDPSSNTVLVSYARWPTDAAQNGDRIKPWMPNGIFYSVYDVASGNWQAPIDVTDQVKERSFQIAGWGGSELYRRNTSLNSQQDWQSNAKIRIVDGAANQIQVADGSRKYVVTLSIDESGGLVANLNGVSAPIILQSEHAKVHSFHDYELQYSALNHTTTLFVDGQQITTWAGEVSQENNIQFGNADAQIDGRLHVQKIVLTQQGHNLVEFDAFYLAQQTPEVEKDLEKLGWTKIKTGNTMSLYGNASVNPGPGHGITLTRQQNISGSQNGRLIYPAIVLDRFFLNVMSIYSDDGGSNWQTGSTLPIPFRWKSSSILETLEPSEADMVELQNGDLLLTARLDFNQIVNGVNYSPRQQFLSKDGGITWSLLEANNANVFSNISTGTVDASITRFEQSDGSHFLLFTNPQGNPAGTNGRQNLGLWFSFDEGVTWKGPIQLVNGASAYSDIYQLDSENAIVIVETDNSNMRILRMPITLLKQKLTLSQN

SEQ ID NO: 2 - protein, 176 aa, Vibrio cholerae
ALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNT

SEQ ID NO: 3 - protein, 1035 aa, Streptococcus pneumoniae
MSYFRNRDIDIERNSMNRSVQERKCRYSIRKLSVGAVSMIVGAVVFGTSPVLAQEGASEQPLANETQLSGESSTLTDTEKSQPSSETELSGNKQEQERKDKQEEKIPRDYYARDLENVETVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSQLFKRSDLEKKLPEGAALTEKTDIFESGRNGKPNKDGIKSYRIPALLKTDKGTLIAGADERRLHSSDWGDIGMVIRRSEDNGKTWGDRVTITNLRDNPKASDPSIGSPVNIDMVLVQDPETKRIFSIYDMFPEGKGIFGMSSQKEEAYKKIDGKTYQILYREGEKGAYTIRENGTVYTPDGKATDYRVVVDPVKPAYSDKGDLYKGNQLLGNIYFTTNKTSPFRIAKDSYLWMSYSDDDGKTWSAPQDITPMVKADWMKFLGVGPGTGIVLRNGPHKGRILIPVYTTNNVSHLNGSQSSRIIYSDDHGKTWHAGEAVNDNRQVDGQKIHSSTMNNRRAQNTESTVVQLNNGDVKLFMRGLTGDLQVATSKDGGVTWEKDIKRYPQVKDVYVQMSAIHTMHEGKEYIILSNAGGPKRENGMVHLARVEENGELTWLKHNPIQKGEFAYNSLQELGNGEYGILYEHTEKGQNAYTLSFRKFNWDFLSKDLISPTEAKVKRTREMGKGVIGLEFDSEVLVNKAPTLQLANGKTARFMTQYDTKTLLFTVDSEDMGQKVTGLAEGAIESMHNLPVSVAGTKLSNGMNGSEAAVHEVPEYTGPLGTSGEEPAPTVEKPEYTGPLGTSGEEPAPTVEKPEYTGPLGTAGEEAAPTVEKPEFTGGVNGTEPAVHEIAEYKGSDSLVTLTTKEDYTYKAPLAQQALPETGNKESDLLASLGLTAFFLGLFTLGKKREQ

SEQ ID NO: 4 - protein, 185 aa, Streptococcus pneumoniae
FMRGLTGDLQVATSKDGGVTWEKDIKRYPQVKDVYVQMSAIHTMHEGKEYIILSNAGGPKRENGMVHLARVEENGELTWLKHNPIQKGEFAYNSLQELGNGEYGILYEHTEKGQNAYTLSFRKFNWDFLSKDLISPTEAKVKRTREMGKGVIGLEFDSEVLVNKAPTLQLANGKTARFMTQYDTK

SEQ ID NO: 5 - protein, 438 aa, Pseudomonas aeruginosa
MNTYFDIPHRLVGKALYESYYDHFGQMDILSDGSLYLIYRRATEHVGGSDGRVVFSKLEGGIWSAPTIVAQAGGQDFRDVAGGTMPSGRIVAASTVYETGEVKVYVSDDSGVTWVHKFTLARGGADYNFAHGKSFQVGARYVIPLYAATGVNYELKWLESSDGGETWGEGSTIYSGNTPYNETSYLPVGDGVILAVARVGSGAGGALRQFISLDDGGTWTDQGNVTAQNGDSTDILVAPSLSYIYSEGGTPHVVLLYTNRTTHFCYYRTILLAKAVAGSSGWTERVPVYSAPAASGYTSQVVLGGRRILGNLFRETSSTTSGAYQFEVYLGGVPDFESDWFSVSSNSLYTLSHGLQRSPRRVVVEFARSSSPSTWNIVMPSYFNDGGHKGSGAQVEVGSLNIRLGTGAAVWGTGYFGGIDNSATTRFATGYYRVRAWI

SEQ ID NO: 6 - protein, 106 aa, Pseudomonas aeruginosa
VPDFESDWFSVSSNSLYTLSHGLQRSPRRVVVEFARSSSPSTWNIVMPSYFNDGGHKGSGAQVEVGSLNIRLGTGAAVWGTGYFGGIDNSATTRFATGYYRVRAWI

SEQ ID NO: 7 - protein, 489 aa, Streptococcus pneumoniae
GAMVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGGSGVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSGGALGVPDFESDWFSVSSNSLYTLSHGLQRSPRRVVVEFARSSSPSTWNIVMPSYFNDGGHKGSGAQVEVGSLNIRLGTGAAVWGTGYFGGIDNSATTRFATGYYRVRAWI

SEQ ID NO: 8 - protein, 489 aa, synthetic construct
  REGION 1..489: note = Modified CBM
GAMVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGGSGVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGSLGVPDFESDWFDVSSNSLYTLSHGLQRSPRRVVVEFARSSSPSTWNIVMPSYFNDGGHKGSGAQVEVGSLNIKLGTGAAVWGTGYFGGIDNSATTRFATGYYRVRAWI

SEQ ID NO: 9 - protein, 489 aa, synthetic construct
  REGION 1..489: note = Modified CBM
GAMVIEKEDVETNASNGQRVDLSSELDKLKKLENATVHMEFKPDPKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGGSGVIEKEDVETNASNGQRVDLSSELDKLKKLENATVHMEFKPDPKAPAFYNLFSVSSATKKDEYFTMAVYNNTATLEGRGSDGKQFYNNYNDAPLKVKPGQWNSVTFTVEKPTAELPKGRARLYVNGGLSRTSLRSGNFIKDMPDVTHVQIGATKRANNTVWGSNLQIRNLTVYNRALTPEEVQKRSGGSLGVPDFESDWFDVSSNSLYTLSHGLQRSPRRVVVEFARSSSPSTWNIVMPSYFNDGGHKGSGAQVEVGSLNIKLGTGAAVWGTGYFGGIDNSATTRFATGYYRVRAWI

SEQ ID NO: 10 - protein, 393 aa, synthetic construct
  REGION 1..393: note = Vc2CBM
GAMALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDALNGSMALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGD

SEQ ID NO: 11 - protein, 789 aa, synthetic construct
  REGION 1..789: note = Vc4CBM
GAMALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDALNGSMALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDLQALGMALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDGGNSGMALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGD

SEQ ID NO: 12 - protein, 188 aa, Streptococcus pneumoniae
GSMVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRS

SEQ ID NO: 13 - protein, 5 aa, synthetic construct
  REGION 1..5: note = Peptide linker
  SITE 3: note = MISC_FEATURE - x = any amino acid
ALXGS

SEQ ID NO: 14 - protein, 5 aa, synthetic construct
  REGION 1..5: note = Peptide linker
LQALG

SEQ ID NO: 15 - protein, 5 aa, synthetic construct
  REGION 1..5: note = Peptide linker
  SITE 3: note = MISC_FEATURE - x = any amino acid
GGXSG

SEQ ID NO: 16 - protein, 5 aa, synthetic construct
  REGION 1..5: note = Peptide linker
GGALG

SEQ ID NO: 17 - protein, 5 aa, synthetic construct
  REGION 1..5: note = Peptide linker
GGGGS

SEQ ID NO: 18 - protein, 10 aa, synthetic construct
  REGION 1..10: note = Peptide linker
  SITE 3: note = MISC_FEATURE - x = any amino acid
ALXGSGGGSG

SEQ ID NO: 19 - protein, 10 aa, synthetic construct
  REGION 1..10: note = Peptide linker
LQALGGGGSL

SEQ ID NO: 20 - protein, 15 aa, synthetic construct
  REGION 1..15: note = Peptide linker
  SITE 3: note = MISC_FEATURE - X = any amino acid
ALXGSGGGSGGGGSG

SEQ ID NO: 21 - protein, 10 aa, Streptococcus pneumoniae
GVLSRTSLRS

SEQ ID NO: 22 - protein, 10 aa, Pseudomonas aeruginosa
WFSVSSNSLY

SEQ ID NO: 23 - protein, 9 aa, Pseudomonas aeruginosa
LSHGLQRSP

SEQ ID NO: 24 - protein, 9 aa, Pseudomonas aeruginosa
GSLNIRLGT

SEQ ID NO: 25 - protein, 12 aa, Streptococcus pneumoniae
FYNLFSVSSATK

SEQ ID NO: 26 - protein, 13 aa, Streptococcus pneumoniae
VRLYVNGVLSRTS

SEQ ID NO: 27 - protein, 15 aa, Streptococcus pneumoniae
KGRVRLYVNGVLSRT

SEQ ID NO: 28 - protein, 15 aa, Pseudomonas aeruginosa
GAQVEVGSLNIRLGT

SEQ ID NO: 29 - protein, 15 aa, Streptococcus pneumoniae
FYNLFSVSSATKKDE

SEQ ID NO: 30 - protein, 15 aa, Pseudomonas aeruginosa
SDWFSVSSNSLYTLS

SEQ ID NO: 31 - protein, 9 aa, Streptococcus pneumoniae
IRNLTVYNR

(19) United States
(12) Patent Application Publication
(10) Pub. No.: US 2024/0115659 A1
(43) Pub. Date: Apr. 11, 2024

(54) VIRAL TREATMENT

(71) Applicant: Pneumagen Ltd., Fife (GB)

(72) Inventors: Helen Connaris, St. Andrews (GB); Lei Yang, Fife (GB); Jane Alexandra Potter, Fife (GB)

(21) Appl. No.: 18/485,455

(22) Filed: Oct. 12, 2023

Related U.S. Application Data
(63) Continuation of application No. 15/733,545, filed on Aug. 25, 2020, now Pat. No. 11,819,534.

(30) Foreign Application Priority Data
Feb. 27, 2018 (GB) 1803197.1

(51) Int. Cl. A61K 38/16; A61K 38/47; A61P 31/12

(52) U.S. Cl. CPC A61K 38/164; A61K 38/47; A61P 31/12

(57) ABSTRACT

Both Sp2CBMTD and Vc2CBMTD were shown to reduce the apical viral load of RSV-infected human airway epithelial cells. The degree of reduction was dependent on the type of mCBM and number of doses administered. Exposure of infected MucilAlr inserts to Sp2CBMTD did not show a significant effect on viral replication due to variability of data but a slight reduction was observed at the highest dose (100 μg) after two doses at 48 h p.i (see FIG. 23A), and after three doses at 96 h p.i. compared to virus infected control. Exposure to Vc2CBMTD (100 μg) was more effective in reducing RSV viral replication in both dosing regimens than using a 10 μg dosing regimen. Although not statistically significant, the reduction of viral load at 48 h p.i was observed to decreased greater than 1 log (FIG. 23B).

### RELATED APPLICATIONS

This application is a continuation of U.S. patent application Ser. No. 15/733,545, filed Aug. 25, 2020, which is a 35 U.S.C. § 371 national stage application of PCT Application No. PCT/GB2019/050546, filed on Feb. 27, 2019, which claims priority from United Kingdom Patent Application No. 1803197.1, filed on Feb. 27, 2018, the contents of each of which are incorporated herein by reference in their entireties. The above-referenced PCT International Application was published in the English language as International Publication No. WO 2019/166802A1 on Sep. 6, 2019.

### STATEMENT REGARDING ELECTRONIC FILING OF A SEQUENCE LISTING

A Sequence Listing in XML format, entitled PG448125USA-sequence listing.xml, 42 kilobytes in size, generated on Oct. 12, 2023, and filed herewith, is hereby incorporated by reference in its entirety for its disclosures.

### FIELD OF THE INVENTION

The invention provides molecules for use in compositions, medicaments and methods for the treatment or prevention of RSV infections, its symptoms and associated pathologies and potentially infections caused or contributed to by viral pathogens which do not bind or do not primarily bind sialic acid containing receptors during pathogenesis.

### BACKGROUND OF THE INVENTION

Respiratory syncytial virus (RSV) is a syncytial virus that causes respiratory tract infections. It causes disease in humans and animals and is a major cause of lower respiratory tract infections and the young and elderly are particularly at risk. Infection with RSV (which includes the form often referred to as human RSV (HRSV)) does induce protective immunity but research shows that the response is not prolonged (or sustained) and multiple instances of infection are not uncommon.

Unlike many other respiratory pathogens, RSV does not bind to sialoglycoconjugates for cell entry and infection³. However, due to the ubiquitous and predominant nature of sialic acid as the terminal glycan of respiratory epithelial cell surface glycoconjugates, it would be advantageous if sialic acid binding molecules could be exploited as the basis of treatments for pathogens such as RSV.

### SUMMARY OF THE INVENTION

The present disclosure is based on the finding that molecules with affinity for (or an ability to bind to) sialic acid (and in particular sialoglycoconjugates) on cell surfaces (these including sialic acid containing glycoproteins and cell surface sialic acid receptors), find utility in the treatment and/or prevention of symptoms, infections, diseases and/or conditions associated with respiratory syncytial virus (RSV).

The present disclosure provides a sialic acid binding molecule for use in the treatment and/or prevention of symptoms, infections, diseases and/or conditions associated with respiratory syncytial virus (RSV).

Further provided is the use of a sialic acid binding molecule in the manufacture of a medicament for use in the treatment and/or prevention of symptoms, infections, diseases and/or conditions associated with RSV.

The disclosure also provides a method of treating or preventing a RSV symptom, disease, infection or condition, said method comprising administering a subject in need thereof a therapeutically effective amount of a sialic acid binding molecule.

The disclosure also provides sialic acid binding molecules and medicaments and methods comprising sialic acid binding molecules, for use in methods of treating or preventing a symptom, disease and/or condition associated with a RSV infection.

Throughout this specification, the terms “comprise”, “comprising” and/or “comprises” is/are used to denote aspects and embodiments of this invention that “comprise” a particular feature or features. It should be understood that this/these terms may also encompass aspects and/or embodiments which “consist essentially of” or “consist of” the relevant feature or features.

RSV is a medium-sized (120-200 nm) enveloped virus containing a lipoprotein coat and a linear negative-sense RNA genome. The genome encodes the F, G, and SH lipoproteins. The F (fusion) and G (attachment) lipoproteins (or glycoproteins) target the host cell membrane and control the initial phases of infection. They are also highly conserved among RSV isolates. Specifically, the G protein targets the ciliated cells of the airways, and the F protein causes the virion membrane to fuse with a target cell membrane. Based on the reactivity of the virus with monoclonal antibodies against the attachment (G) and fusion (F) glycoproteins, RSV is divided into two antigenic subgroups, A and B. As used herein, the term “RSV” includes all strains, forms and antigenic variants of RSV—including all forms, strains and variants of human (H) or animal RSV.

RSV is not a pathogen which binds to or associates with sialic acid during pathogenesis; thus RSV does not bind host cell sialic acid (or cell surface sialoglycoconjugates). Accordingly, the finding that sialic acid binding molecules (such as, for example, CBM40 molecules (and others defined below)) have a utility in the treatment of viral pathogens which do not exploit cell surface sialic acid (sialoglycoconjugates) during pathogenesis (or pathogens in which sialic acid binding is not a primary means by which the pathogens binds to, colonises or enters/infects a cell), is wholly unexpected.

For prior art disclosures where sialic acid binding molecules have been used as agents capable of blocking the binding of pathogens to cell surface sialic acid/sialoglycoconjugates, the utility of the sialic acid binding molecule is rooted in the fact that both the sialic acid binding molecule and the pathogen bind sialic acid; this is not the case with RSV. Because RSV does not bind sialic acid, one of skill would not appreciate that a sialic acid binding molecule could be used to block RSV entry into a cell.

Without wishing to be bound by theory, it is suggested that when bound to cell surface sialic acid/sialoglycoconjugates, sialic acid binding molecules prevent the G and/or F RSV proteins from accessing their targets on the host cell. This, in turn, prevents the RSV from colonising and infecting the cell. Further, data presented below (see in particular the data presented in Example 6) shows that the RSV viral surface comprises glycoproteins which terminate with sialic acid; as such, it is suggested that sialic acid binding molecules are able to bind to these glycoproteins (via the sialic acid component) and further inhibit binding between the RSV and the host cell.

While it may have been established that sialic acid binding proteins can be used to prime or modulate the immune system, and that a primed or modulated immune response may impact on the pathology of a whole host of different pathogens (including those that do not bind or. which do not primarily bind sialic acid during pathogenesis), one of skill would still not have been led to the finding that molecules with an affinity for sialic acid (for example. CBMs such as CBM40 type molecules) can be used to physically block, prevent or neutralise RSV infection.

The observation that sialic acid binding molecules can nevertheless be used to neutralise or block a RSV infection extends this disclosure to the provision of sialic acid binding molecules for use in neutralising or blocking of a RSV infection. Such uses may be applied to in vitro or in vivo methods.

The disclosure further provides the use of a sialic acid binding molecule for the manufacture of medicaments for neutralising or blocking a RSV infection.

The disclosure also relates to a method of neutralising or blocking a RSV infection, said method comprising administering a therapeutically effective amount of a sialic acid binding molecule to a subject in need thereof.

Alternatively, the disclosure provides methods that may be used to render cells non-permissive to RSV (the term “non-permissive” meaning a cell which resists viral attachment or colonisation and/or subsequent infection and viral replication). Such methods (which methods may be in vitro or in vivo methods) may comprise contacting or incubating cells susceptible or vulnerable to RSV infection with a sialic acid binding molecule described herein. The step of contacting or incubating may comprise contacting or incubating a cell with a sialic acid binding molecule prior to contact with RSV. Additionally or alternatively, the step of contacting or incubating a cell with a sialic acid binding molecule may be extended so that the sialic acid binding molecule is contacted with the cell at the same time as the cell is in contact with RSV.

Without wishing to be bound by theory, during the period of incubation, sialic acid containing cell surface receptors will be bound by the sialic acid binding molecule and although RSV does not itself bind sialic acid moieties (sialoglycoconjugates) on the cell surface, it has surprisingly been found that binding between sialic acid binding molecules and cell surface sialic acid containing receptors/sialoglycoconjugates, inhibits or prevents RSV cell binding. This, in turn, prevents RSV cell infection and intracellular RSV replication.

The findings reported in this disclosure have important implications for the formulation and administration of molecules with sialic acid binding affinity and for the subsequent use of these formulations in the treatment and/or prevention of RSV infections. For example, the finding that sialic acid binding molecules (for example, CBMs and/or CBM40 type molecules) can be used to prevent (or neutralise) RSV infection allows for the use of sialic acid binding molecules as formulations suitable for mucosal administration.

Sialic acid binding molecule containing formulations for mucosal administration may be used to
(i) prevent RSV binding to host cells; and/or
(ii) block or neutralise RSV.

As stated, the term “block” or “neutralise” refers to the blocking or neutralising effect of the sialic acid binding molecule which binds to host cell sialic acid containing receptors/sialoglycoconjugates and which has now (surprisingly) been found to block RSV cell binding (and subsequent infection).

Accordingly, the disclosure provides a composition for mucosal administration, said composition comprising a sialic acid binding molecule for use in the treatment and/or prevention of diseases and/or conditions associated with RSV.

It should be noted that the term “mucosal administration” embraces compositions that have been formulated for administration to any mucosal surface, including, for example, respiratory surfaces and the like. The term also embraces compositions formulated for administration by inhalation. Compositions suitable (or formulated) for mucosal administration may include compositions, which are intended to be administered intranasally.

Thus a composition for mucosal administration may be formulated with excipients, diluents and/or buffers which are suitable for use in any type of mucosal administration.

Compositions for mucosal (for example intranasal) administration may comprise solutions of the sialic acid binding molecule(s) to be administered and/or particles (comprising the same) for aerosol dispersion or dispensed in drinking water. When dispensed such compositions should desirably have a particle diameter in the range 10 to 200 microns to enable retention in, for example, the nasal cavity; this may be achieved by, as appropriate, use of a powder of a suitable particle size or choice of an appropriate valve. Other suitable compositions include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension. A composition for mucosal administration may be provided in the form of a liquid spray.

In one embodiment, the disclosure may not relate to the treatment and/or prevention of RSV by the priming or modulation of an immune response.

The disclosure may further provide sialic acid binding molecules for prophylactic use. Specifically, the sialic acid molecules described herein may be used prophylactically in order to prevent a RSV infection.

A method of prophylaxis or of preventing a RSV infection may comprise administering a subject in need thereof a composition of this disclosure. As stated, a composition of this disclosure may comprise a composition formulated for mucosal (for example intranasal) administration.

As used herein and in any method of preventing a RSV infection, a “subject in need thereof” may be any subject predisposed, susceptible or at risk of developing a RSV infection. The subject may be a neonate, an infant or a child. The subject may be elderly. The subject may be an immunocompromised subject. The subject may have one or more underlying or chronic health problems—in particular, problems affecting the respiratory tract and/or respiration. For example, the subject may suffer from asthma.

Throughout this specification, we use the term “sialic acid binding molecule” this term embraces any useful sialic acid binding molecule. Useful sialic acid binding molecules may take any form and/or belong to any class of molecule or compound (for example they may be proteins, peptides, carbohydrates, antibodies and the like) and the term “sialic acid” embraces all forms of N- or O-substituted neuraminic acid and includes all synthetic, naturally occurring and/or modified forms thereof. Sialic acids may be found as components of cell surface molecules, glycoproteins and glycolipids. Most often, sialic acids are present at the end (terminal regions) of sugar chains connected to cell membranes and/or proteins. For example, some cells of the human upper respiratory tract comprise α-2,6-linked sialic acid receptors and other cells of the upper and lower respiratory tracts comprise α-2,3-linked sialic acid receptors. The sialic acid family encompasses a number (approximately 50) of derivatives that may result from acetylation, glycolylation, lactonisation and methylation at C4, C5, C7, C8 and C9. All such derivatives are to be embraced by the term “sialic acid”.

Furthermore, sialic acids are found linked α(2,3) or α(2,6) to GaI and GaINAc or α(2,8) or α(2,9) to another sialic acid. Accordingly, it is important to understand that while the term “sialic acid” is used throughout this specification, it encompasses all derivatives, analogues or variants (either naturally occurring or synthetically generated) thereof as well as monomers, dimers, trimers, oligomers, polymers or concatamers comprising the same.

Thus, a sialic acid binding molecule of this disclosure (and for use as described herein) comprises a moiety which exhibits an affinity for sialic acid—including all forms of sialic acid described above and any form of sialic acid present on the surface of a cell (perhaps as part of a cell surface receptor), for example a mammalian cell. These various forms of sialic acid may be collectively referred to as “sialic acid moieties”.

The sialic acid binding molecules of this disclosure exhibit an affinity for sialic acid and as such they may bind/couple to and/or associate with one or more sialic acid moieties. Thus, the term “sialic acid binding molecule” may further encompass any fragment of a whole sialic acid binding molecule which retains an ability to bind to or otherwise couple or associate with a sialic acid moiety.

Sialic acid binding molecules for use may comprise a single sialic acid binding molecule (a monomeric or monovalent molecule, for example) or, alternatively, two or more sialic acid binding molecules—which two or more molecules may be the same or different—a polymeric or multivalent molecule, for example.

A sialic acid binding molecule for use may comprise, consist essentially of or consist of, one or more of the sialic acid binding molecules known as “carbohydrate binding modules” (CBMs). CBMs suitable for use exhibit an affinity for sialic acid. CBMs are classified into families and CBMs classed as members of the family 40 CBMs (CBM40) may be useful. The family 40 CBMs embrace molecules of approximately 200 residues and are often found at the N-terminus of GH33 sialidases. They may also be found inserted in the β-propeller of GH33 sialidases.

The disclosure may embrace the use of molecules, for example, larger molecules, which comprise a sialic acid binding component. As stated, that sialic acid binding component (i.e. the sialic acid binding molecule) may itself comprise (consist of or consist essentially of) a CBM, for example, a CBM40. By way of (non-limiting) example, the molecules (e.g. the sialic acid binding molecules) of this disclosure may not only exhibit an ability to bind sialic acid, but may also have one or more other functions. For example, the molecules may have enzymatic activity. For example, a useful molecule may comprise a CBM (as described herein) and exhibit some sialidase activity.

A useful sialic acid binding molecule may be a fusion protein comprising an enzymatic portion and a sialic acid binding portion—wherein the sialic acid binding portion comprises a CBM as described herein. In such cases, the enzymatic portion may be fused to the sialic acid binding portion. As stated, the enzymatic portion of any useful fusion protein may comprise (or have, or exhibit) sialidase activity.

In one embodiment, the sialic acid binding protein or CBM for the various uses described herein, may not be provided as part of, or comprised within, a molecule (for example a fusion protein) with enzymatic (for example sialidase) activity. Additionally or alternatively, the sialic acid binding molecule may not (i) bind heparin or heparin sulfate and/or (ii) comprise the GAG-binding domain of a protein that binds heparin or heparin sulfate moieties.

As such, the present disclosure provides a CBM or CBM40 for use in the treatment and/or prevention of diseases and/or conditions associated with respiratory syncytial virus (RSV).

Further provided is the use of a CBM or CBM40 in the manufacture of a medicament for use in the treatment and/or prevention of diseases and/or conditions associated with RSV.

The disclosure also provides a method of treating or preventing a RSV infection, said method comprising administering a subject in need thereof a therapeutically effective amount of a CBM or CBM40.

The disclosure also provides sialic acid binding molecules and medicaments and methods comprising a CBM or CBM40 for use in methods of treating or preventing a disease and/or condition associated with a RSV infection.

This disclosure also provides CBM or CBM40 for use in neutralising or blocking a RSV infection.

The disclosure further provides the use of a CBM and/or CBM40 for the manufacture of medicaments for neutralising or blocking a RSV infection.

The disclosure relates to a method of neutralising or blocking a RSV infection, said method comprising administering a therapeutically effective amount of a CBM and/or CBM40 to a subject in need thereof.

Alternatively, the disclosure provides methods that may be used to render cells non-permissive to RSV (the term “non-permissive” meaning a cell which resists viral attachment or colonisation and/or subsequent infection and viral replication). Such methods (which methods may be in vitro or in vivo methods) may comprise contacting or incubating cells susceptible or vulnerable to RSV infection with a CBM and/or CBM40 of this disclosure.

CBM and/or CBM40 containing formulations for mucosal administration may be used to
(i) prevent RSV binding to host cells; and/or
(ii) block or neutralise RSV.

The disclosure provides a composition for mucosal administration, said composition comprising a CBM and/or CBM40 for use in the treatment and/or prevention of diseases and/or conditions associated with RSV. As stated, a composition for mucosal administration may be formulated with excipients, diluents and/or buffers which are suitable for use in any type of mucosal administration.

The disclosure may further provide a CBM or CBM40 for prophylactic use. Specifically, CBM or CBM40 described herein may be used prophylactically in order to prevent a RSV infection.

As stated, a composition of this disclosure may comprise a composition formulated for mucosal (for example, intranasal) administration.

Exemplary carbohydrate binding modules (CBMs) for use may comprise the sialic acid binding domain of Vibrio cholerae NanH sialidase (VcCBM: a CBM40) and/or the equivalent (or homologous) domain from Streptococcus pneumoniae NanA sialidase (SpCBM: also a CBM40). Of course, similar or homologous sialic acid binding modules present in other organisms are to be encompassed within the scope of the term “CBM”.

An exemplary Vibrio cholerae NanH sialidase amino acid sequence is deposited under accession umber A5F7A4 and is reproduced below as SEQ ID NO: 1 (781 amino acids).

MRFKNVKKTA LMLAMFGMAT SSNAALFDYN ATGDTEFDSP
AKQGWMQDNT NNGSGVLTNA DGMPAWLVQG IGGRAQWTYS
LSTNQHAQAS SFGWRMTTEM KVLSGGMITN YYANGTQRVL
PIISLDSSGN LVVEFEGQTG RTVLATGTAA TEYHKFELVF
LPGSNPSASF YFDGKLIRDN IQPTASKQNM IVWGNGSSNT
DGVAAYRDIK FEIQGDVIFR GPDRIPSIVA SSVTPGVVTA
FAEKRVGGGD PGALSNTNDI ITRTSRDGGI TWDTELNLTE
QINVSDEFDF SDPRPIYDPS SNTVLVSYAR WPTDAAQNGD
RIKPWMPNGI FYSVYDVASG NWQAPIDVTD QVKERSFQIA
GWGGSELYRR NTSLNSQQDW QSNAKIRIVD GAANQIQVAD
GSRKYVVTLS IDESGGLVAN LNGVSAPIIL QSEHAKVHSF
HDYELQYSAL NHTTTLFVDG QQITTWAGEV SQENNIQFGN
ADAQIDGRLH VQKIVLTQQG HNLVEFDAFY LAQQTPEVEK
DLEKLGWTKI KTGNTMSLYG NASVNPGPGH GITLTRQQNI
SGSQNGRLIY PAIVLDRFFL NVMSIYSDDG GSNWQTGSTL
PIPFRWKSSS ILETLEPSEA DMVELQNGDL LLTARLDFNQ
IVNGVNYSPR QQFLSKDGGI TWSLLEANNA NVFSNISTGT
VDASITRFEQ SDGSHFLLFT NPQGNPAGTN GRQNLGLWFS
FDEGVTWKGP IQLVNGASAY SDIYQLDSEN AIVIVETDNS
NMRILRMPIT LLKQKLTLSQ N

The CBM region of SEQ ID NO: 1 is from amino acid residue 25 to 216—this sequence may be SEQ ID NO: 2.

An exemplary Streptococcus pneumoniae NanA sialidase amino acid sequence has been deposited under accession number P62575 and is reproduced below as SEQ ID NO: 3 (1035 amino acids).

MSYFRNRDID IERNSMNRSV QERKCRYSIR KLSVGAVSMI
VGAVVFGTSP VLAQEGASEQ PLANETQLSG ESSTLTDTEK
SQPSSETELS GNKQEQERKD KQEEKIPRDY YARDLENVET
VIEKEDVETN ASNGQRVDLS SELDKLKKLE NATVHMEFKP
DAKAPAFYNL FSVSSATKKD EYFTMAVYNN TATLEGRGSD
GKQFYNNYND APLKVKPGQW NSVTFTVEKP TAELPKGRVR
LYVNGVLSRT SLRSGNFIKD MPDVTHVQIG ATKRANNTVW
GSNLQIRNLT VYNRALTPEE VQKRSQLFKR SDLEKKLPEG
AALTEKTDIF ESGRNGKPNK DGIKSYRIPA LLKTDKGTLI
AGADERRLHS SDWGDIGMVI RRSEDNGKTW GDRVTITNLR
DNPKASDPSI GSPVNIDMVL VQDPETKRIF SIYDMFPEGK
GIFGMSSQKE EAYKKIDGKT YQILYREGEK GAYTIRENGT
VYTPDGKATD YRVVVDPVKP AYSDKGDLYK GNQLLGNIYF
TTNKTSPFRI AKDSYLWMSY SDDDGKTWSA PQDITPMVKA
DWMKFLGVGP GTGIVLRNGP HKGRILIPVY TTNNVSHLNG
SQSSRIIYSD DHGKTWHAGE AVNDNRQVDG QKIHSSTMNN
RRAQNTESTV VQLNNGDVKL FMRGLTGDLQ VATSKDGGVT
WEKDIKRYPQ VKDVYVQMSA IHTMHEGKEY IILSNAGGPK
RENGMVHLAR VEENGELTWL KHNPIQKGEF AYNSLQELGN
GEYGILYEHT EKGQNAYTLS FRKFNWDFLS KDLISPTEAK
VKRTREMGKG VIGLEFDSEV LVNKAPTLQL ANGKTARFMT
QYDTKTLLFT VDSEDMGQKV TGLAEGAIES MHNLPVSVAG
TKLSNGMNGS EAAVHEVPEY TGPLGTSGEE PAPTVEKPEY
TGPLGTSGEE PAPTVEKPEY TGPLGTAGEE AAPTVEKPEF
TGGVNGTEPA VHEIAEYKGS DSLVTLTTKE DYTYKAPLAQ
QALPETGNKE SDLLASLGLT AFFLGLFTLG KKREQ

The CBM region of SEQ ID NO: 3 is from amino acid residue 121 to 305—this sequence may be SEQ ID NO: 4.

Thus, CBMs for use as sialic acid binding molecules in the various aspects and embodiments of this disclosure may comprise a protein or peptide having the sequence of SEQ ID NO: 1, 2, 3 or 4 or a sequence fragment derived therefrom and which encodes a molecule with an ability to bind sialic acid (in other words a sialic acid binding molecule encoding portion of fragment of SEQ ID NOS: 1, 2, 3 or 4).

A useful sialic acid binding molecule may comprise a proteinaceous moiety encoded by the sialic acid binding domain of the nanH gene (encoding sialidase) of V. cholerae (as provided by SEQ ID NO: 1) or an equivalent or homologous gene present in another organism (for example the equivalent/homologous nanA sialidase gene of S. pneumoniae: see SEQ ID NO: 3).

A sialic acid binding molecule for use may comprise from about residue 1, 5, 10, 15, 25 or 30 (i.e. from 1-30 or from any amino acid residue there between) to about residue 150, 175, 200, 210, 216, 220-781 (to any residue from 150 to 781 including any residue therebetween) of the V. cholerae sialidase molecule of SEQ ID NOS: 1 and 2. For example a sialic acid binding molecule for use may comprise a peptide having a sequence corresponding to residue 25 to about residue 216 of SEQ ID NO: 1 above.

A further suitable sialic acid binding molecule may comprise a protein or peptide having the sequence of SEQ ID NO: 3 or 4 or a sialic acid binding fragment thereof. For example, a useful sialic acid binding molecule may comprise a proteinaceous moiety encoded by the sialic acid binding domain of the Streptococcus pneumoniae nanA gene (encoding sialidase). For example a sialic acid binding molecule for use may comprise from about residue 80, 90, 100, 110, 120, 121 to 130 (i.e. from any of about residues 80 to 130 including any residue therebetween) to about residue 250, 275, 300, 305, 310, 320-1035 (i.e. to any residue from about 250-1035 including to about any residue therebetween) of the S. pneumoniae sialidase molecule of SEQ ID NOS: 3 and 4. For example, a sialic acid binding molecule for use may comprise a peptide having a sequence corresponding to residue 121 to about residue 305 of SEQ ID NO: 3 above.

A sialic acid binding molecule for use may comprise one or more CBMs. For example, suitable sialic acid binding molecules may comprise single CBMs—for example a single VcCBM or a single SpCBM. Alternatively, a sialic acid binding molecule for use may comprise a plurality or multiple (i.e. two or more) CBMs. Sialic acid binding molecules which comprise a plurality of CBMs may be termed “multivalent sialic acid binding molecules” or “multivalent CBMs”. A multivalent CBM may, for example, comprise two or more (for example three, four, five or six) VcCBMs or two or more SpCBMs. A multivalent CBM may comprise a mixture of different CBMs, for example one or more VcCBMs with one or more SpCBMs.

The sialic acid binding molecules for use may further comprise an oligomerisation domain. Suitable oligomerisation domains may exhibit an ability to self-associate to form multimer structures, for example trimers. An oligomerisation domain for use may comprise any molecule with the above mentioned oligomerisation properties or any functional fragment thereof. For example, one or more (for example two) sialic acid binding molecules (for example CBMs) may be bound, coupled or fused to an oligomerisation domain—the resulting sialic acid binding molecule::oligomerisation domain “fusion” may then be used (with one or more other such “fusions”) as a molecule for modulating cell growth and/or activity and/or for treating or preventing any of the diseases and/or conditions disclosed herein.

Suitable oligomerisation domains may be derived from, for example, Pseudomonas aeruginosa pseudaminidase. An exemplary Pseudomonas aeruginosa pseudaminidase sequence amino acid sequence has been deposited under accession number PAO579 and is reproduced below as SEQ ID NO: 5 (438 amino acids).

MNTYFDIPHR LVGKALYESY YDHFGQMDIL SDGSLYLIYR
RATEHVGGSD GRVVFSKLEG GIWSAPTIVA QAGGQDFRDV
AGGTMPSGRI VAASTVYETG EVKVYVSDDS GVTWVHKFTL
ARGGADYNFA HGKSFQVGAR YVIPLYAATG VNYELKWLES
SDGGETWGEG STIYSGNTPY NETSYLPVGD GVILAVARVG
SGAGGALRQF ISLDDGGTWT DQGNVTAQNG DSTDILVAPS
LSYIYSEGGT PHVVLLYTNR TTHFCYYRTI LLAKAVAGSS
GWTERVPVYS APAASGYTSQ VVLGGRRILG NLFRETSSTT
SGAYQFEVYL GGVPDFESDW FSVSSNSLYT LSHGLQRSPR
RVVVEFARSS SPSTWNIVMP SYFNDGGHKG SGAQVEVGSL
NIRLGTGAAV WGTGYFGGID NSATTRFATG YYRVRAWI

The oligomerisation domain of SEQ ID NO: 5 is from amino acid residue 333 to 438—this sequence may be SEQ ID NO: 6.

Thus an oligomerisation domain for use may comprise from about residue 250, 275, 300, 310, 320, 333, 340 to 350 (i.e. from about residue 250 to about residue 350 including from about any residue therebetween) to about residue 400, 410, 420, 430 or 438 (i.e. to about any residue from about residue 400 residue 438 including to about any residue therebetween) of the P. aeruginosa pseudaminidase trimerisation domain (PaTD) provided by SEQ ID NO: 5. For example, a useful sialic acid binding molecule may exploit an oligomerisation domain comprising residues 333 to 438 of SEQ ID NO: 6.

A sialic acid binding molecule for use may comprise one or more of the CBM based molecules presented in FIG. 1. For example, a suitable sialic acid binding molecule may comprise (consist essentially of, or consist of) two or more VcCBMs optionally fused, bound or conjugated to an oligomerisation domain (such as a PaTD or oligomerisation fragment thereof). The sialic acid binding molecule may comprise, consist or consist essentially of two fused (or bound) VcCBMs which are, in turn, fused to an oligomerisation domain (see, for example, molecule Vc2CBMTD shown in FIG. 1).

Other sialic acid binding domains for use may comprise two or more SpCBMs optionally fused, bound or conjugated to an oligomerisation domain (such as a PaTD or an oligomerisation fragment thereof). Sialic acid binding molecules for use may comprise, consist or consist essentially of two fused (or bound) SpCBMs which are in turn fused to an oligomerisation domain (see, for example, molecule Sp2CBMTD shown in FIG. 1).

The terms “sialic acid binding molecule”, “CBM” and/or “CBM40” may include modified forms of any of the molecules described herein. Further, the term “modified” embraces molecules which contain one or more mutations relative to a reference sequence.

A “reference sequence” may be any wild type CBM sequence. For example, a reference sequence may comprise, consist essentially of or consist of a wild type family 40 CBM sequence, e.g. the wild type CBM sequences from Vibrio cholerae NanH sialidase or Streptococcus pneumoniae NanA sialidase (it should be appreciated that similar or homologous CBMs (including CBM40s) present in other organisms are to be encompassed within the scope of the term “CBM” and/or as CBM reference sequences). A reference sequence from which a useful sialic acid binding molecule may be derived (including useful multivalent CBMs as described herein) may comprise any of the specific sequences described herein (for example SEQ ID NO: 1, 2, 3, 4 and 5.

For example, a modified CBM sequence for use may be derived from a specific or particular wild type CBM. A useful modified CBM sequence may comprise a wild type CBM sequence which includes one or more mutations.

The one or more mutation(s) may be functional. The mutations may, for example, alter the overall primary sequence of a CBM for use, but may not (substantially) alter the properties of the CBM—thus, while the sequence of a modified CBM may be different from the wild-type sequence from which it is derived, the overall function of the modified CBM is (substantially) identical to that of the wild-type CBM. Alternatively, the one or more mutation(s) may individually (and/or independently) or collectively (for example synergistically) modulate (improve or suppress/inhibit) one or more of the physiological, biological immunological and/or pharmacological properties characteristic of a wild type CBM (for example the wild type CBM from which the modified CBM is derived). In particular, the one or more mutations may:
(i) alter the immunogenicity (or antigenicity) of the CBM; and/or
(ii) alter (for example improve) the efficacy (of the CBM or of any multimeric molecule comprising a modified CBM)' and/or
(iii) they may modulate (for example improve) the thermostability of the CBM; and/or
(iv) they may modulate (for example improve) the solubility of the CBM; and/or
(v) they may modulate (for example improve) the in vivo half-life of the molecule.

A “mutation” may include any alteration to a wild-type CBM molecule. For example, the term “mutation” may embrace, for example:
(i) one or more amino acid substitution(s) (where one or more of the wild type amino acid(s) is/are swapped or changed for another (different) amino acid—the term “substitutions” would include conservative amino acid substitutions); and/or
(ii) one or more amino acid deletion(s) (where one or more of the wild type amino acid residue(s) are removed); and/or
(iii) one or more amino acid addition(s)/insertion(s) (where additional amino acid residue(s) are added to a wild type (or reference) primary sequence); and/or
(iv) one or more amino acid/sequence inversions (usually where two or more consecutive amino acids in a primary sequence are reversed; and/or
(v) one or more amino acid/sequence duplications (where an amino acid or a part of the primary amino acid sequence (for example a stretch of 5-10 amino acids) is repeated)

Thus, a useful modified CBM (i.e. a CBM for use in the medical uses and methods described herein) may comprise one or more of the mutations described herein.

By way of non-limiting example, the following represent individual units (referred to as “HEX” units) which may be used to make hexameric sialic acid binding molecules for use in the various embodiments described herein (for example for use in methods of treating or preventing RSV infections etc.). In each case, the HEX unit comprises two modified CBMs (denoted CBM1 and CBM2) with the specific mutations introduced to each CBM being identified in parenthesis. It should be noted that a “ [symbol] ” symbol indicates an amino acid linker (linking one CBM to another or a CBM to an oligomerisation domain). As such, a hexameric sialic acid binding molecule may be made up of several (for example 3) HEX units. In each case, the oligomerisation domain (denoted “TD”) conjugates the units together as a trimer. While any given hexamer may comprise identical copies of the units described above (and below under the headings HEX1, HEX2, HEX3, HEX4, HEX5, HEX6 and HEX17), one of skill will appreciate that further options are available. For example, a HEX unit may be made up of two CBMs, each having different mutations (the mutations being one or more selected from the options detailed herein).

### (i) HEX1

CBM1 (L170T V239A V246G I286A Y292E) [symbol] CBM2 (L170T V239A V246G I286A Y292E) [symbol] TD (S342D L348D R403K)

### (ii) HEX2

CBM1 (V239A V246G I286A Y292E) [symbol] CBM2 (V239A V246G I286A Y292E) [symbol] TD (S342D R403K)

(iii) HEX3

CBM1 (V239A V246G I286A) [symbol] CBM2 (V239A V246G I286A) [symbol] TD (S342D R403K)

### (iv) HEX4

CBM1 (V239A V246G) [symbol] CBM2 (V239A V246G) [symbol] TD (S342D)

### (v) HEX5

CBM1 (V239A V246G) [symbol] CBM2 (V239A V246G) [symbol] TD (R403K)

### (vi) HEX6

CBM1 (V239A V246G) [symbol] CBM2 (V239A V246G) [symbol] TD (S342D R403K)

(vii) HEX17

CBM1 (V239A V246G A162P) [symbol] CBM2 (V239A V246G A162P) [symbol] TD (S342D R403K)

It will be noted that HEX6 and HEX17 are identical except for the additional A162P mutation. This proline mutation (a substitution for the wild type Alanine at residue 162) has been shown to improve thermostability (the single CBM Tm by 3-4° C.). Further information regarding the use of proline mutations may be derived from Fu 2009, ‘Increasing protein stability by improving beta-turns’ (DOI 10.1002/prot.22509) which describes the general approach. The proline mutation does not affect (increase or decrease) the predicted immunogenicity of the CBM molecule, is not located near the other mutations, the N- or C-termini or the ligand binding site. Rather unexpectedly, beyond the modest improvement in thermostability, it was noted that the A162P mutation yields hexameric CBMs (i.e. a molecule comprising 3×HEX17 units) exhibiting a marked improvement in in vivo experiments—in particular in comparison to those same experiments conducted using a hexameric molecule comprising 3×Hex6 units. For example, the modified molecules (in particular a molecule comprising 3×HEX17 units) exhibit modulation over pro-inflammatory cytokines, including for example IL-8. Indeed the modulatory effect (specifically an inhibitory effect) on the production of IL-8 by a molecule comprising 3×HEX17 units, was improved over other tested modified molecules.

Relative to the amino acid sequences of Sp2CBMTD (aka “SpOrig” SEQ ID NO: 7) the amino acid sequence of the HEX6 (SEQ ID NO: 8) and HEX 17 (SEQ ID NO: 9) molecules is:

| | |
|---|---|
| SpOrig | GAMVIEKEDVETNASNGQRVDLSSELDKLKKLENATVHMEFKPDAKAPAFYNLFSVSSAT |
| HEX6 | GAMVIEKEDVETNASNGQRVDLSSELDKLKKLENATVHMEFKPDAKAPAFYNLFSVSSAT |
| Hex17 | GAMVIEKEDVETNASNGQRVDLSSELDKLKKLENATVHMEFKPDPKAPAFYNLFSVSSAT |
| SpOrig | KKDEYFTMAVYNNTATLEGRGSDGKQFYNNYNDAPLKVKPGQWNSVTFTVEKPTAELPKG |
| HEX6 | KKDEYFTMAVYNNTATLEGRGSDGKQFYNNYNDAPLKVKPGQWNSVTFTVEKPTAELPKG |
| Hex17 | KKDEYFTMAVYNNTATLEGRGSDGKQFYNNYNDAPLKVKPGQWNSVTFTVEKPTAELPKG |
| SpOrig | RVRLYVNGVLSRTSLRSGNFIKDMPDVTHVQIGATKRANNTVWGSNLQIRNLTVYNRALT |
| HEX6 | RARLYVNGGLSRTSLRSGNFIKDMPDVTHVQIGATKRANNTVWGSNLQIRNLTVYNRALT |
| Hex17 | RARLYVNGGLSRTSLRSGNFIKDMPDVTHVQIGATKRANNTVWGSNLQIRNLTVYNRALT |
| SpOrig | PEEVQKRSGGGSGVIEKEDVETNASNGQRVDLSSELDKLKKLENATVHMEFKPDAKAPAF |
| HEX6 | PEEVQKRSGGGSGVIEKEDVETNASNGQRVDLSSELDKLKKLENATVHMEFKPDAKAPAF |
| Hex17 | PEEVQKRSGGGSGVIEKEDVETNASNGQRVDLSSELDKLKKLENATVHMEFKPDPKAPAF |
| SpOrig | YNLFSVSSATKKDEYFTMAVYNNTATLEGRGSDGKQFYNNYNDAPLKVKPGQWNSVTFTV |
| HEX6 | YNLFSVSSATKKDEYFTMAVYNNTATLEGRGSDGKQFYNNYNDAPLKVKPGQWNSVTFTV |
| Hex17 | YNLFSVSSATKKDEYFTMAVYNNTATLEGRGSDGKQFYNNYNDAPLKVKPGQWNSVTFTV |
| SpOrig | EKPTAELPKGRVRLYVNGVLSRTSLRSGNFIKDMPDVIHVQIGATKRANNTVWGSNLQIR |
| HEX6 | EKPTAELPKGRARLYVNGGLSRTSLRSGNFIKDMPDVTHVQIGATKRANNTVWGSNLQIR |
| Hex17 | EKPTAELPKGRARLYVNGGLSRTSLRSGNFIKDMPDVTHVQIGATKRANNTVWGSNLQIR |
| SpOrig | NLTVYNRALTPEEVQKRSGGALGVPDFESDWFSVSSNSLYTLSHGLQRSPRRVVVEFARS |
| HEX6 | NLIVYNRALTPEEVQKRSGGSLGVPDFESDWFDVSSNSLYILSHGLQRSPRRVVVEFARS |
| Hex17 | NLTVYNRALTPEEVQKRSGGSLGVPDFESDWFDVSSNSLYTLSHGLQRSPRRVVVEFARS |
| SpOrig | SSPSTWNIVMPSYFNDGGHKGSGAQVEVGSLNIRLGTGAAVWGTGYFGGIDNSATTRFAT |
| HEX6 | SSPSTWNIVMPSYFNDGGHKGSGAQVEVGSLNIKLGTGAAVWGTGYFGGIDNSATTRFAT |
| Hex17 | SSPSTWNIVMPSYFNDGGHKGSGAQVEVGSLNIKLGTGAAVWGTGYFGGIDNSATTRFAT |
| SpOrig | GYYRVRAWI |
| HEX6 | GYYRVRAWI |
| Hex17 | GYYRVRAWI |

Thus, the various aspects and embodiments of this disclosure (uses, sialic acid binding molecules for use, methods and medicaments) may exploit sialic acid binding molecules which comprise, consist of or consist essentially of sialic acid binding molecules selected from the group consisting of:
(i) one or more VcCBM(s);
(ii) one or more SpCBM(s);
(iii) one or more modified CBM(s);
(iv) a HEX17 molecule; and
(iii) a multivalent CBM.

As such, the present disclosure provides:
Sp2CBM;
HEX17;
Vc2CBM; and/or
Vc4CBM;
for use in the treatment and/or prevention of a RSV infection.

Further provided is the use of Vc2CBM or Vc4CBM in the manufacture of a medicament for use in the treatment and/or prevention of a RSV infection.

The disclosure also provides the use of HEX17 in the manufacture of a medicament for use in the treatment and/or prevention of a RSV infection.

The disclosure also relates to a method of treating or preventing RSV, said method comprising the steps of administering to a subject in need thereof, a therapeutically effective amount of
HEX17;
Sp2CBM;
Vc2CBM; and/or
Vc4CBM.

For the avoidance of doubt, HEX17 is a multivalent modified CBM as described above. Vc2CBM comprises, consists essentially of or consists of two Vibrio cholerae NanH sialidase CBM units linked, bound or conjugated together. An exemplary Vc2CBM sequence may comprise, consist essentially of or consist of:

(SEQ ID NO: 10)
GAMALFDYNATGDTEFDSPAKQGWMQDNINNGSGVLINADGMPAWLVQGI
GGRAQWTYSLSTNQHAQASSFGWRMTTEMKVLSGGMITNYYANGTQRVLP
IISLDSSGNLVVEFEGQTGRTVLATGTAATEYHKFELVFLPGSNPSASFY
FDGKLIRDNIQPTASKQNMIVWGNGSSNTDGVAAYRDIKFEIQGDALNGS
MALFDYNATGDTEFDSPAKQGWMQDNINNGSGVLINADGMPAWLVQGIGG
RAQWTYSLSTNQHAQASSFGWRMTTEMKVLSGGMITNYYANGTQRVLPII
SLDSSGNLVVEFEGQTGRTVLATGTAATEYHKFELVFLPGSNPSASFYFD
GKLIRDNIQPTASKQNMIVWGNGSSNTDGVAAYRDIKFEIQGD

Vc4CBM comprises, consists essentially of or consists of four Vibrio cholerae NanH sialidase CBM units linked, bound or conjugated together. An exemplary Vc4CBM sequence may comprise, consist essentially of or consist of the following sequence:

(SEQ ID NO: 11)
GAMALFDYNATGDTEFDSPAKQGWMQDNINNGSGVLINADGMPAWLVQGI
GGRAQWTYSLSTNQHAQASSFGWRMTTEMKVLSGGMITNYYANGTQRVLP
IISLDSSGNLVVEFEGQTGRTVLATGTAATEYHKFELVFLPGSNPSASFY
FDGKLIRDNIQPTASKQNMIVWGNGSSNTDGVAAYRDIKFEIQGDALNGS
MALFDYNATGDTEFDSPAKQGWMQDNINNGSGVLINADGMPAWLVQGIGG
RAQWTYSLSTNQHAQASSFGWRMITEMKVLSGGMITNYYANGTQRVLPII
SLDSSGNLVVEFEGQTGRTVLATGTAATEYHKFELVFLPGSNPSASFYFD
GKLIRDNIQPTASKQNMIVWGNGSSNTDGVAAYRDIKFEIQGDLQALGMA
LFDYNATGDTEFDSPAKQGWMQDNINNGSGVLINADGMPAWLVQGIGGRA
QWTYSLSTNQHAQASSFGWRMTTEMKVLSGGMITNYYANGTQRVLPIISL
DSSGNLVVEFEGQTGRTVLATGTAATEYHKFELVFLPGSNPSASFYFDGK
LIRDNIQPTASKQNMIVWGNGSSNTDGVAAYRDIKFEIQGDGGNSGMALF
DYNATGDTEFDSPAKQGWMQDNINNGSGVLINADGMPAWLVQGIGGRAQW
TYSLSTNQHAQASSFGWRMTTEMKVLSGGMITNYYANGTQRVLPIISLDS
SGNLVVEFEGQTGRTVLATGTAATEYHKFELVFLPGSNPSASFYFDGKLI
RDNIQPTASKQNMIVWGNGSSNTDGVAAYRDIKFEIQGD

Sp2CBM comprises, consists essentially of or consists of two Streptococcus pneumoniae NanA sialidase units linked, bound or conjugated together. An exemplary Sp2CBM sequence may comprise, consist essentially of, or consist of two copies of the following sequence:

(SEQ ID NO: 12)
GSMVIEKEDVETNASNGQRVDLSSELDKLKKLENATVHMEFKPDAKAPAF
YNLFSVSSATKKDEYFTMAVYNNTATLEGRGSDGKQFYNNYNDAPLKVKP
GQWNSVTFTVEKPTAELPKGRVRLYVNGVLSRTSLRSGNFIKDMPDVTHV
QIGATKRANNTVWGSNLQIRNLTVYNRALTPEEVQKRS

The two copies of the above mentioned sequence may be joined via any one of the peptide linker sequences described herein. For example, a Sp2CBM sequence may comprise, consist essentially of, or consist of

GSMVIEKEDVETNASNGQRVDLSSELDKLKKLENATVHMEFKPDAKAPAF
YNLFSVSSATKKDEYFTMAVYNNTATLEGRGSDGKQFYNNYNDAPLKVKP
GQWNSVTFIVEKPTAELPKGRVRLYVNGVLSRTSLRSGNFIKDMPDVTHV
QIGATKRANNTVWGSNLQIRNLTVYNRALTPEEVQKRS[xxxxx][xxxx
xxxxxx][xxxxxxxxxxxxxxx]GSMVIEKEDVETNASNGQRVDLSSEL
DKLKKLENATVHMEFKPDAKAPAFYNLFSVSSATKKDEYFTMAVYNNTAT
LEGRGSDGKQFYNNYNDAPLKVKPGQWNSVTFTVEKPTAELPKGRVRLYV
NGVLSRTSLRSGNFIKDMPDVTHVQIGATKRANNTVWGSNLQIRNLTVYN
RALTPEEVQKRS

Wherein [xxxxx], [xxxxxxxxxx] and [xxxxxxxxxxxxxxx]—represent the choice of linker peptide sequences as outlined below. Both CBM sequences would be joined by one of the 5, 10 or 15 amino acid linker sequences described herein.

Vc2CBM and Vc4CBM may be described as tandem-repeat multivalent proteins based on the Family 40 sialic acid binding domain (CBM) of the nanH gene encoding the sialidase from V. cholerae. Sp2CBM may be described as a tandem-repeat multivalent protein based on the family 40 sialic acid binding domain (CBM) of the nanA gene encoding the sialidase from S. pneumoniae.

The disclosed molecules (for the uses and methods described herein) may be generated using PCR-based cloning techniques and a suitable method for the generation of multivalent molecules of this type is described in, for example, Connaris et al, 2009 (Enhancing the Receptor Affinity of the Sialic Acid-Binding Domain of Vibrio cholerae Sialidase through Multivalency; J. Biol. Chem; Vol. 284(11); pp 7339-7351). For example, multivalent CBM molecules, including the likes of HEX17, Vc2CBM, Vc4CBM and Sp2CBM may be prepared as constructs comprising multiple CBMs linked by amino acid/peptide linkers. Each CBM (for example VcCBM) may be linked to another by, for example, peptides comprising 5, 10 or 15 amino acids. By way of example any one or more of the following peptides may be used to link two or more CBMs to produce a multivalent CBM:

| | |
|---|---|
| (i) 5 amino acid linkers: | ALXGS |
| | (SEQ ID NO: 13) |
| | LQALG |
| | (SEQ ID NO: 14) |
| | GGXSG |
| | (SEQ ID NO: 15) |
| | GGALG |
| | (SEQ ID NO: 16) |
| | GGGGS |
| | (SEQ ID NO: 17) |
| (ii) 10 amino acid linkers: | ALXGSGGGSG |
| | (SEQ ID NO: 18) |
| | LQALGGGGSL |
| | (SEQ ID NO: 19) |
| (iii) 15 amino acid linkers: | ALXGSGGGSGGGGSG |
| | (SEQ ID NO: 20) |

where “X” is any amino acid.

An exemplary Vc4CBM may take the following form:

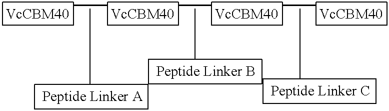

This schematic shall be referred to hereinafter as General Formula 1.

Thus, a Vc4CBM molecule may conform to General Formula 1 as set out above, wherein Peptide Linkers A, B and/or C are selected from the linker options presented above as (i), (ii) and/or (iii). It should be noted that the term “VcCBM40” embraces not only the complete family 40 CBM derived from Vibrio cholerae (NanH sialidase) but also sialic acid binding fragments derived therefrom. Indeed, each of the VcCBM units shown in General Formula 1 may be selected from the group consisting of:
(i) a Vibrio cholerae NanH sialidase CBM; and
(ii) a Vibrio cholerae NanH sialidase CBM sialic acid binding fragment thereof.

Thus, each of the VcCBM units of the molecule shown in General Formula 1 may be the same or different.

Further, it should be noted that the various uses, methods and medicaments described herein may exploit one or more of the sialic acid binding molecules described herein. For example, two or more different sialic acid binding molecules may be administered to a subject together, concurrently or separately.

The present disclosure may provide compositions for use in the various uses, medicaments and methods described herein. As such, any of the sialic acid binding molecule(s) described herein may be formulated for use. For example, a sialic acid binding molecule (or molecules) may be formulated as therapeutic or pharmaceutical compositions. The various compositions may comprise one or more of the sialic acid binding molecules described herein and any given treatment may require the administration (together, concurrently or separately) of one or more of these compositions.

Pharmaceutical compositions according to the present invention, in particular those formulations for mucosal or intranasal administration may be prepared conventionally, comprising substances that are customarily used in pharmaceuticals and as described in, for example, Remington's The Sciences and Practice of Pharmacy, 22nd Edition (Pharmaceutical Press 2012) and/or Handbook of Pharmaceutical Excipients, 7th edition (compiled by Rowe et al, Pharmaceutical Press, 2012)—the entire content of all of these documents and references being incorporated by reference.

Any suitable amount of a sialic acid binding molecule (for example, any of the CBM type molecules described herein, including the CBM40s) may be used. For example, whether a composition comprising a sialic acid binding molecule (for example a CBM such as Sp2CBM or Vc2CBMTD) is to be administered intravenously or mucosally (for example, intranasally) the dose of sialic acid binding molecule may comprise anywhere between about 0.1 μg and about 1000 μg. For example, a dose of about (for example +/−0.5 μg) 0.1 μg, 0.5 μg, 1 μg, 5 μg, 10 μg, 11 μg, 12 μg, 13 μg, 14 μg, 15 μg, 20 μg, 30 μg, 40 μg, 50 μg, 100 μg, 200 μg, 300 μg, 400 μg, 500 μg, 600 μg, 700 μg, 800 μg, 900 μg or 950 μg of the sialic acid binding molecule may be used. These amounts may be provided in any suitable volume of excipient, diluent or buffer. For example, the amount of sialic acid binding molecule may be provided in anywhere between about 1 μl to about 5 ml of excipient, diluent or buffer. For example, the required amount of sialic acid binding molecule may be combined (or formulated) with about 5 μl, 10 μl, 15 μl, 20 μl, 25 μl, 30 μl, 35 μl, 40 μl, 45 μl, 50 μl, 55 μl, 60 μl, 65 μl, 70 μl, 75 μl, 80 μl, 85 μl, 90 μl, 95 μl, 100 μl, 200 μl, 300 μl, 400 μl, 500 μl, 600 μl, 700 μl, 800 μl, 900 μl, 1 ml, 2 ml, 3 ml or 4 ml. Concentrations of 0.1-1 mg (sialic acid binding protein) per ml (excipient, diluent or buffer) may be most useful.

### DETAILED DESCRIPTION

### Brief Description of the Drawings

The present invention will now be described in detail with reference to the following figures which show:

FIG. 1: Building blocks of the multivalent CBM forms and their affinities for sialic acid. a, VcCBM, residues 25-216 of the V. cholerae sialidase (PDB:1w0p) with α-2,3-sialyllactose drawn as spheres. b, SpCBM, residues 121-305 of S. pneumoniae NanA sialidase with α-2,3-sialyllactose (PDB:4c1w). c, TD, the trimerisation domain, residues 333-438, of the P. aeruginosa pseudaminidase (PDB:2w38) in rainbow colours; the other two monomers in single colors. d, Multivalent forms: their molecular weights, valencies and binding affinities for α2,3-sialyllactose as determined by surface plasmon resonance (SPR) at 25° C. (K_{D} values for VcCBM, Vc2CBM and Vc3CBM had been reported previously⁷). Tandem repeat CBMs, and oligomeric CBMs fused to TD are linked by a 5-amino linker.

FIG. 2. Prophylactic effect of mCBM40s in a RSV-infected mammalian cell line. Hep2:NPro cells were incubated with varying amounts (0.1-100 μg/well) of either Sp2CBMTD or Vc2CBMTD for 1 hour before the addition of RSV (1.5×10³ PFU per well). Absorbance readings of infected cells taken at 24 h, 48 h, 72 h and 90 h post infection for Sp2CBMTD (a-d), and for Vc2CBMTD (e-h) treated cells compared to untreated, infected cells, are shown. Bars indicate the mean absorbance change±SD from six replicates. All values are presented as mean±SD, with statistical results presented as: *p<0.05, **p<0.01, ***p<0.001, and ****p<0.0001.

FIG. 3. Effect of mCBM40s in a RSV-infected mammalian cell line. Hep2:NPro cells were incubated with varying amounts (0.1-100 μg/well) of either Sp2CBMTD or Vc2CBMTD with RSV (1.5×10³ PFU per well) prior to adding to cells. Absorbance readings of infected cells taken at 24 h, 48 h, 72 h and 90 h post infection for Sp2CBMTD (a-d), and for Vc2CBMTD (e-h) treated cells compared to untreated, infected cells, are shown. Bars indicate the mean absorbance change±SD from six replicates. All values are presented as mean±SD, with statistical results presented as: *p<0.05, **p<0.01, ***p<0.001, and ****p<0.0001.

FIG. 4. Therapeutic effect of mCBM40s in a RSV-infected mammalian cell line. Hep2:NPro cells were incubated with RSV (1.5×10³ PFU per well) prior to adding varying amounts (0.1-100 μg/well) of either Sp2CBMTD or Vc2CBMTD at 24 h, 48 h or 72 h post infection. Absorbance readings of infected cells were taken at 90 h post infection for Sp2CBMTD (a-c), and for Vc2CBMTD (d-f) treated cells compared to untreated, infected cells, are shown. Bars indicate the mean absorbance change±SD from six replicates. All values are presented as mean±SD, with statistical results presented as: *p<0.05, **p <0.01, ***p<0.001, and ****p<0.0001.

FIG. 5: ProPred predictions of antigenic peptides. A. SpCBM sequence (SEQ ID NO: 4). B. PaTD sequence (SEQ ID NO: 6). Predicted binders are coloured blue, with the first residue of each binding region shown in red. Antigenic peptides predicted by Nordic Biopharma (green bars) and ProImmune (purple bars) are shown under the sequences.

FIG. 6: Expression test of wild type and mutated domains. Lane 1, M12 standard; Lane 2, WTSpCBM; Lanes 3-11, Im15-Im23; Lanes 12-15, Im24-Im27; Lane 16, WT PaTD. A) Whole cell extracts, B) Soluble extracts.

FIG. 7: Position of peptide 167-181 in the SpCBM structure

FIG. 8: Expression and Ni-NTA pull-down of variants Im28 to Im34

FIG. 9: Sites of the HEX17 mutations on the hexamer structure. The quarternary structure of HEX17 was modelled by assembling the crystal structures of the individual SpCBM (pdb code 4c1x) and PaTD (pdb code 2w38) into the hexamer (i.e. 6 copies of SpCBM and 3 copies of PaTD per molecule). The positions of the bound ligand (α2,3-sialyllactose) are shown in stick form (orange). The positions of the mutations are also shown: Blue, the sites of the A162P mutation; Cyan, the sites of the other two CBM mutations; Magenta, the sites of the TD mutations.

FIG. 10: IL-8 stimulation. A549 cells were stimulated by the addition of 10 μg of biologic (Hex17). Cell supernatant was harvested at 24 or 48 h time-points and the IL-8 content was determined by ELISA. Statistical significance between control and treated cells was determined with one-way ANOVA using Tukey's multiple comparison test.

FIGS. 11A-11F: Multiplex analysis of inflammatory mediators. A549 cells were stimulated by the addition of 10 μg of biologic (Sp2CBMTD (aka SpOrig), HEX6 or HEX17). Cell supernatant was harvested at 6 h, 24 or 48 h time-points and inflammatory mediators analysed using a Human Cytokine 12-plex Assay. Statistical significance between control and/or WT hexamer and hexamer variants was determined using a one-way ANOVA (Tukey's multiple comparison test).

FIG. 12: Percentage survival of CBM-treated and untreated mice when lethally challenged with influenza strain PR8. CBM2, CBM3 and CBM4 represent HEX17, HEX6 and WT (SpOrig) respectively. Single CBM dosed animals were given 100 μg of CBM one day prior to lethal challenge with PR8; repeat dosed animal were given 2×0.1 μg of CBM at day-3 and day-1 prior to PR8 challenge.

FIG. 13: Clinical scores of CBM-treated and untreated mice during PR8 infection. CBM2, CBM3 and CBM4 represent HEX17, HEX6 and WT (SpOrig) respectively. An ascending clinical score of 1 to 5 indicates no symptoms (1) to lethargy and death (5), respectively.

FIG. 14: Percentage weight loss of CBM-treated and untreated mice during PR8 infection. CBM2, CBM3 and CBM4 represent Hex17, Hex6 and WT (SpOrig) respectively.

FIG. 15: Anti-mCBM antibody analysis of Day 21 lung homogenates and sera tissue from a PR8-challenged mouse study. Statistical significance between WT hexamer and hexamer variants was determined using a one-way ANOVA (Tukey's multiple comparison test).

FIGS. 16A and B: ELISA results to show interaction of Sp2CBMTD and Vc2CBMTD with RSV.

FIG. 17A-E: Assay to show binding of GFP-Sp2CBMTD and Vc2CBMTD with RSV. PBS used in control assays.

FIGS. 18A and B: ELISA results to show interaction between Sp2CBMTD and Sp2CBMTD-R274Q (at 0.1 μg and 10 ng) with RSV.

FIGS. 19A and B: ELISA results to show interaction between Sp2CBMTD and Vc2CBMTD with neuraminidase treated RSV.

FIGS. 20A and B: data to show the interaction between 3′-SL treated Sp2CBMTD/Vc2CBMTD and RSV

FIGS. 21A and B: Data to show that mammalian cell (HEp2:NPro cell) protection by CBMs (Sp2CBMTD and Vc2CBMTD) from RSV is decreased after cells are treated using neuraminidase.

FIG. 22 (panels A-F): Effect of mCBM40s in a RSV-infected mammalian cell line. Hep2:NPro cells were incubated with varying amounts (0.1-100 μg/well) of either Vc4CBM or HEX17 with RSV (1.5×10³ PFU per well) prior to adding to cells. Absorbance readings of infected cells taken at 24 h, 48 h, and 72 h post infection for Vc4CBM (A, C and E), and for HEX17 (B, D and F) treated cells compared to untreated, infected cells, are shown. Bars indicate the mean absorbance change±SD from six replicates. All values are presented as mean±SD, with statistical results presented as: *p<0.05, **p<0.01, ***p<0.001, and ****p<0.0001.

FIGS. 23A and B: RSV-B viral genome copy number quantification of CBM-treated infected MucilAir inserts at 48 h p.i. compared to virus-only infected cells. A) Sp2CBMTD, B) Vc2CBMTD.

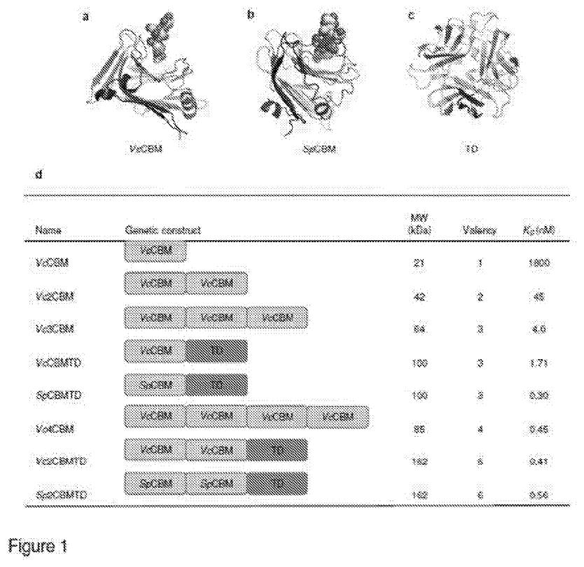

### METHODS AND RESULTS

### Example 1

CBM40 proteins, Sp2CBMTD, Vc2CBMTD and Vc4CBM endotoxin-free) were prepared as described in Connaris et al (2014)¹. Hex17 was prepared as described above. The mammalian cell line HEp2:NPro (a cell line designed to constitutively overexpress BVDV N-terminal protease fragment, Npro that only responds to IFN but not produce IFN) was purchased from ATCC⁴. Cells were maintained in T75 flasks in DMEM/10% FBS (with 1% Penicillin/Streptomycin), and incubated at 37° C. and 5% CO₂. RSV-A2 strain was prepared in SF-DMEM to a dilution of 10⁻², equivalent to 1.5×10⁴ PFU/ml.

For infection studies, 96 well plates (Nunc) were seeded with 100 μL of cells (2.5×10⁵ cells/well) in DMEM/10% FCS and incubated at 37° C. and 5% CO₂ for 24 hrs until cells reached 80-90% confluency. CBM40 proteins (100 μL, 10-fold serial dilution of 10 mg/ml stock in SF-DMEM) were either added to cells before RSV infection (100 μL, 1.5×10³ PFU per well in SF-DMEM), mixed with RSV for 1 hour on ice before adding to cells, or added to cells after RSV infection. In each case, CBM40s and/or RSV were left for 1 hour before removal. Cells were then overlaid with 300 μL AVICEL and plates were left to incubate for either 24, 48, 72, or 90 h at 37° C. and 5% CO₂.

To determine the level of RSV in cells post infection, AVICEL was removed and cells were fixed with 10% PFA in PBS for 10 min at room temperature prior to immunostaining with primary mouse anti-RSV F antibody (1:200 dilution, Serotec) followed by goat anti-mouse HRP IgG antibody (1:2000, Santa Cruz). The presence of RSV was detected using the colorimetric substrate TMB (Sigma). Absorbance was measured at the 450-nm wavelength (620-nm wavelength used as reference).

Statistical Analysis. Group comparisons were made using one-way ANOVA and Dunnett's multiple comparisons test. GraphPad Prism 7 (GraphPad Software) was used for all analysis. Tests with p<0.05 were deemed statistically significant. Unless otherwise stated, all values are presented as mean±SD, with statistical results presented as: *p<0.05, **p<0.01, ***p<0.001, and ****p<0.0001.

### Summary

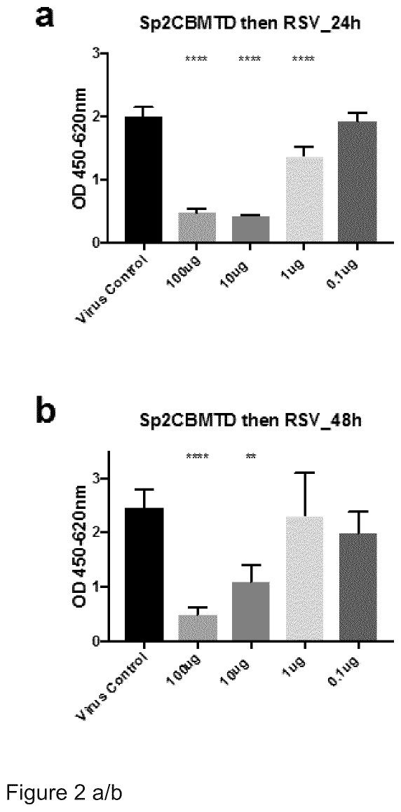

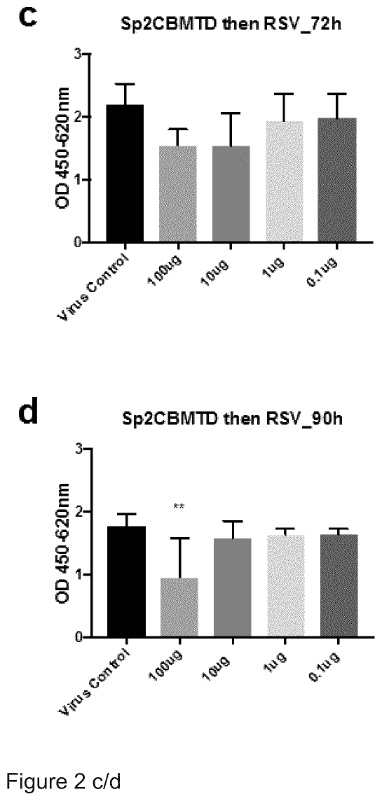

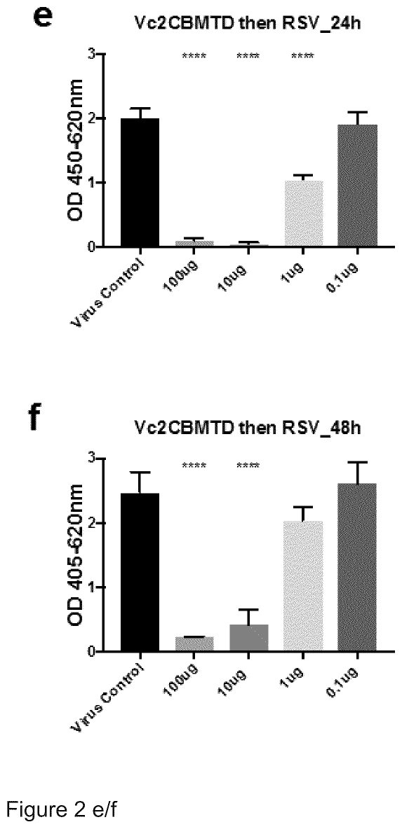

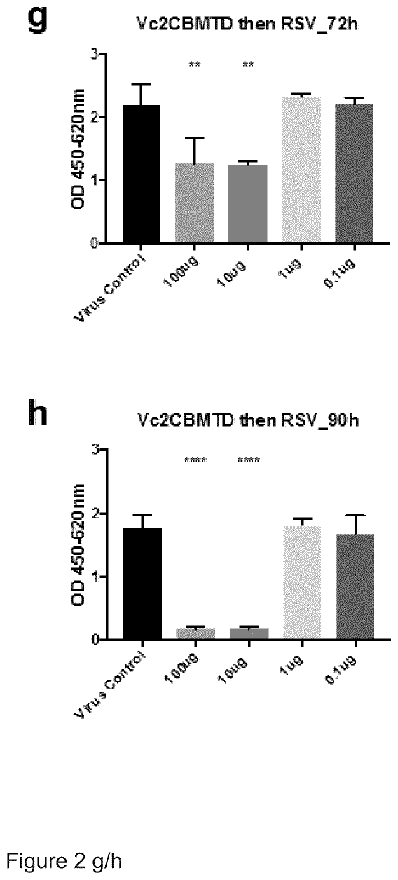

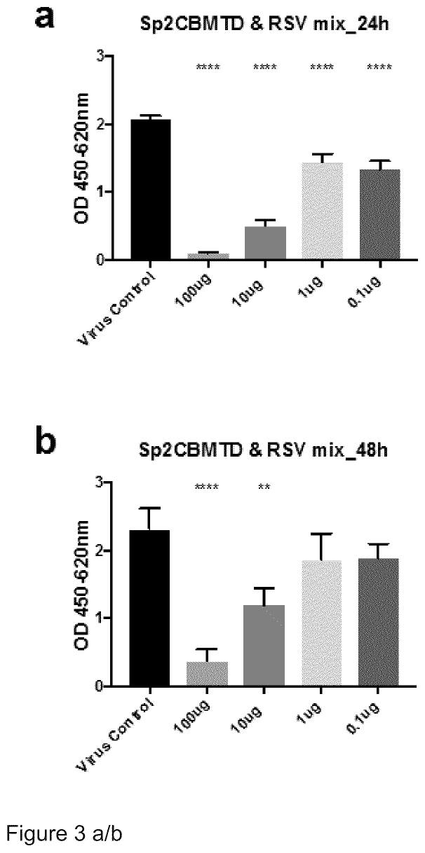

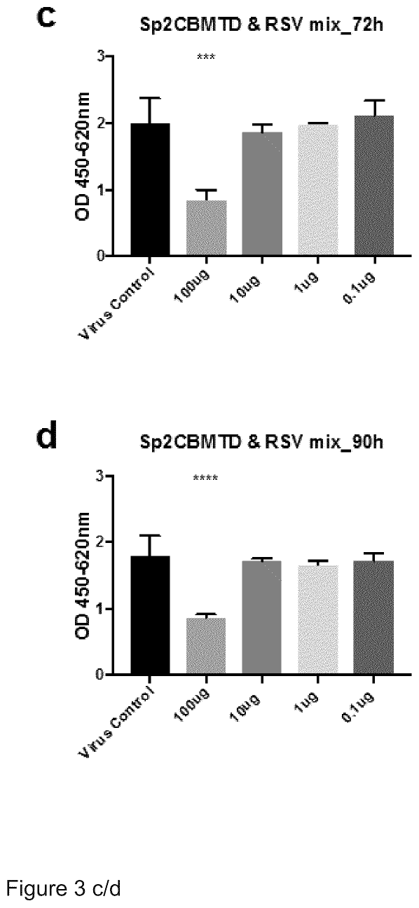

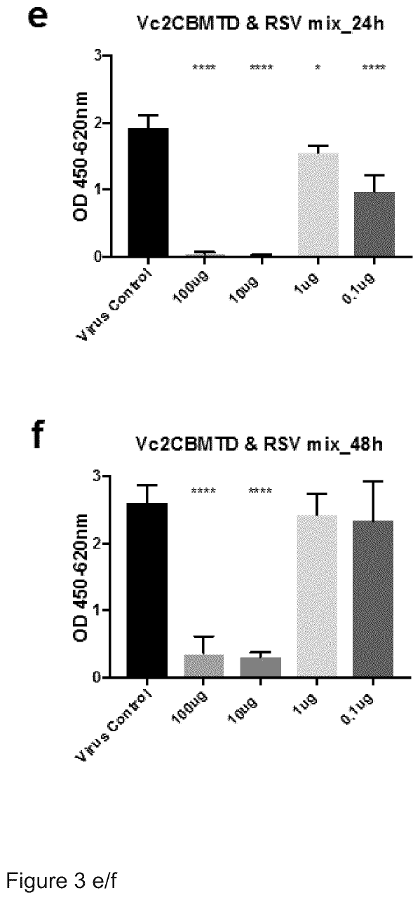

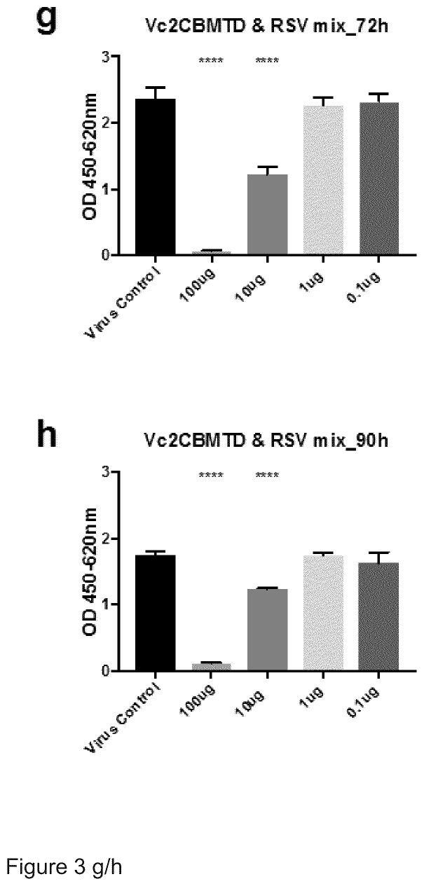

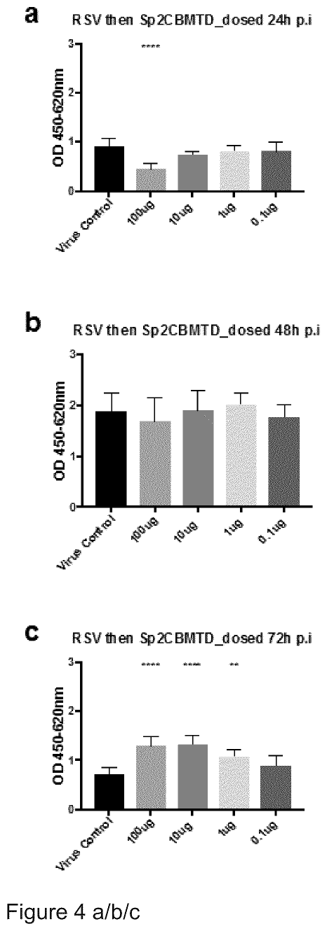

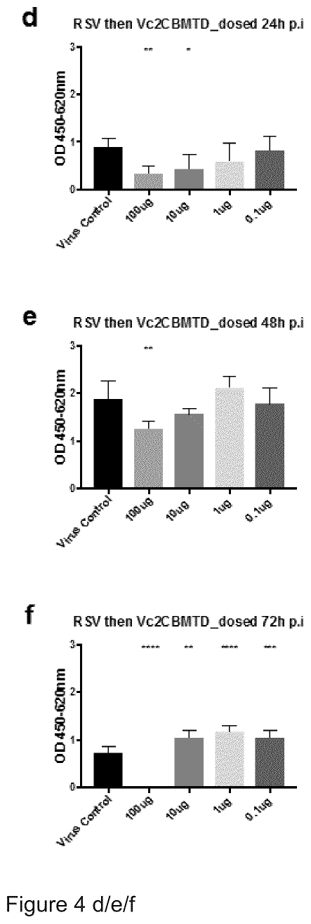

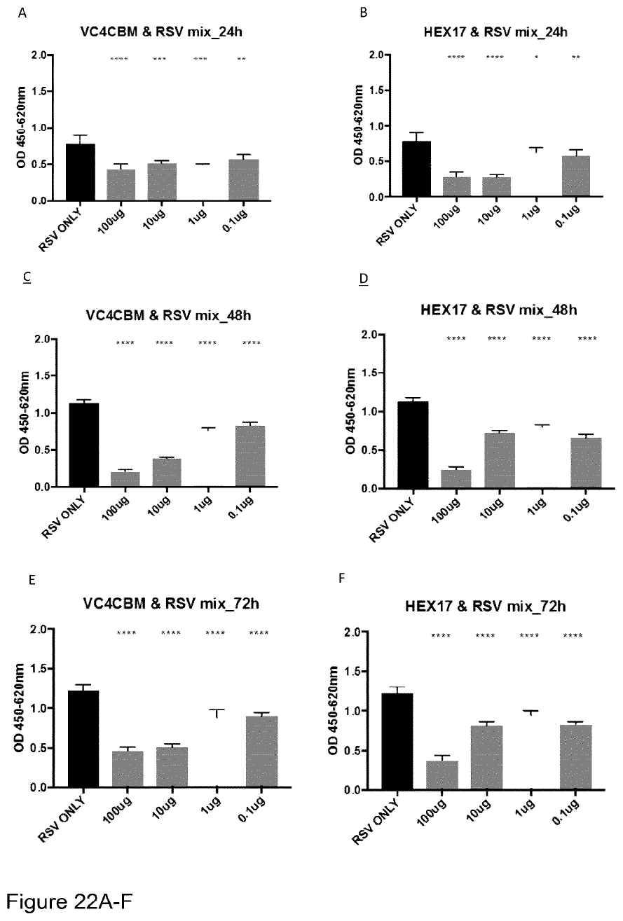

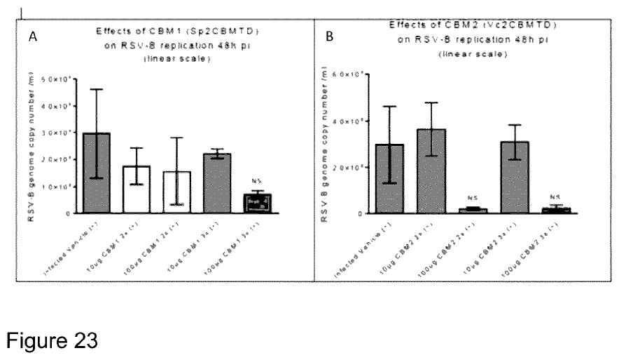

The results are summarized in FIGS. 2, 3, 4 and 22.
1. Single dose administration of mCBM40s, when given prior to, or at the same time, as RSV in a Hep2:Npro mammalian cell line, appeared to reduce or inhibit RSV attachment to cells.
2. When given at the same time with RSV, all mCBM40s, up to 0.1-1 mg/ml concentrations, appeared to have a significant effect in reducing RSV infection compared to prophylactic dosing, when cells were tested up to 90 h post-infection.
3. Dosing of mCBM40s 24 h post RSV infection demonstrated reduction of viral infection when given at the highest dose. The highest dose of Vc2CBMTD when given at 24 h, 48 h and 72 h post RSV administration appeared to beneficially affect RSV levels in the Hep2:Npro mammalian cell line.
4. Of the mCBM40s tested, Vc2CBMTD appeared to be the most effective against RSV infection in the mammalian cell line.

### REFERENCES

[1] Connaris H, Crocker, P R and Taylor, G L (2009). Enhancing the Receptor Affinity of the Sialic Acid-binding Domain of Vibrio cholerae Sialidase through Multivalency. JBC 284: 7339-7351.
[2] Connaris H, Govorkova E A, Ligertwood Y, Dutia B M, Yang L, Tauber S, Taylor M A, Alias N, Hagan R, Nash A A, Webster R G, Taylor G L (2014). Prevention of influenza by targeting host receptors using engineered proteins. Proc Natl Acad Sci USA 111:6401-6406.
[3] Harris, J and Werling, D (2003). Binding and entry of respiratory syncytial virus into host cells and initiation of the innate immune response. Cellular Microbiology 5: 671-680.
[4] Hilton, L., Moganeradj, K., Zhang, G., Chen, Y. H., Randall, R. E., McCauley, J. W., and Goodbourn, S (2006). The NPro product of bovine viral diarrhea virus inhibits DNA binding by interferon regulatory factor 3 and targets it for proteasomal degradation. J Virol 80(23), 11723-32.

### Example 2

### Sp2CBMTD: Prediction of Immunogenic Regions

### Nordic Biopharma in Silico Screen

The in silico T-cell epitope screening identified four significant and two borderline immunogenic clusters:

Significant:

| Domain | Residue range | Sequence |
|---|---|---|
| SpCBM | 245 to 254 | GVLSRTSLRS |
| | | (SEQ ID NO: 21) |
| PaTD | 340 to 349 | WFSVSSNSLY |
| | | (SEQ ID NO: 22) |
| PaTD | 351 to 359 | LSHGLQRSP |
| | | (SEQ ID NO: 23) |
| PaTD | 398 to 406 | GSLNIRLGT |
| | | (SEQ ID NO: 24) |

Borderline:

| Domain | Residue range | Sequence |
|---|---|---|
| SpCBM | 167 to 178 | FYNLFSVSSATK |
| | | (SEQ ID NO: 25) |
| SpCBM | 239 to 251 | VRLYVNGVLSRTS |
| | | (SEQ ID NO: 26) |

### ProImmune Human Donor T-Cell Proliferation Assay

The ProImmune study highlighted two regions of high antigenicity and two regions of moderate antigenicity:

High antigenicity:

| Domain | Residue range | Sequence |
|---|---|---|
| SpCBM | 236 to 250 | KGRVRLYVNGVLSRT |
| | | (SEQ ID NO: 27) |
| PaTD | 392 to 406 | GAQVEVGSLNIRLGT |
| | | (SEQ ID NO: 28) |

Moderate antigenicity:

| Domain | Residue range | Sequence |
|---|---|---|
| SpCBM | 167 to 181 | FYNLFSVSSATKKDE |
| | | (SEQ ID NO: 29) |
| PaTD | 338 to 352 | SDWFSVSSNSLYTLS |
| | | (SEQ ID NO: 30) |

### ProPred in Silico Analysis

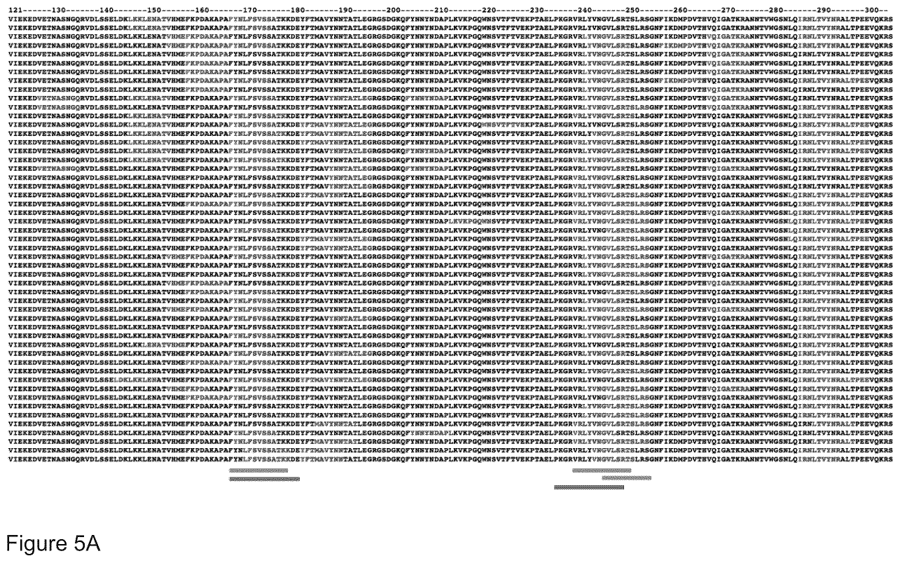

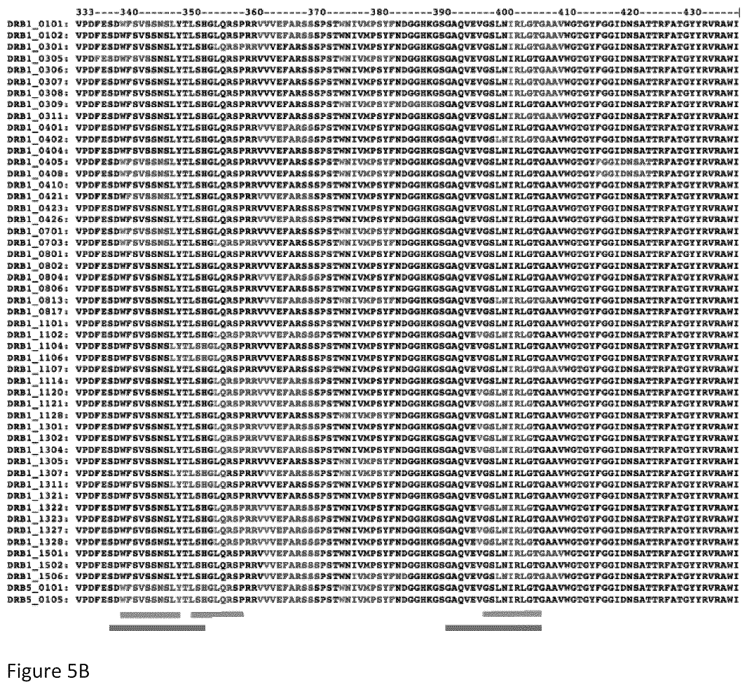

A further in silico tool, the online ProPred server⁴, was also used. The output of the ProPred server is shown in FIG. 5. The relative positions of the Nordic Biopharma/ProImmune epitopes are also highlighted and indicate reasonable agreement between the three methods. In addition to the epitopes listed above, ProPred strongly predicted another immunogenic epitope in the SpCBM domain:

| Domain | Residue range | Sequence |
|---|---|---|
| SpCBM | 286 to 294 | IRNLTVYNR |
| | | (SEQ ID NO: 31) |

### Mutations in the Individual CBM and TD Domains

To guide the design of mutations that might reduce immunogenicity, ProPred was used to test the effect of changing each residue in these peptides to every alternative residue. Those that gave the greatest reduction in predicted number of allele binders were noted. As the crystal structure of both the SpCBM and TD domains are known, these mutations were also modelled to reduce the likelihood of introducing mutations that would obviously disrupt the protein structure.

Initially, nine single mutations in SpCBM and four single mutations in PaTD were introduced and are listed below (‘Im’ is short for immunogenicity mutant):

| (SpCBM) variants | Mutation | (PaTD) variants | Mutation |
|---|---|---|---|
| WTSp | — | WTTD | — |
| Im15 | Y168W | Im24 | S342D |
| Im16 | L170A | Im25 | S345D |
| Im17 | L170T | Im26 | L348D |
| Im18 | V173G | Im27 | R403K |
| Im19 | V239A | | |
| lm20 | V239T | | |
| Im21 | V246G | | |
| Im22 | I286A | | |
| Im23 | Y292E | | |

Note: Im1 to Im14 (not shown) were introduced by mutagenesis into a non-codon optimized background, before the ProImmune data were available.

### Synthesis of WT and Mutated Constructs

The genes encoding WT SpCBM, WT PaTD and the variants Im15 to Im27 were codon optimized for E. coli expression and synthesized by GeneArt. The genes were then cloned in-house into the pHISTEV vector for expression as 6His-tagged proteins.

### Expression and Biophysical Characterization

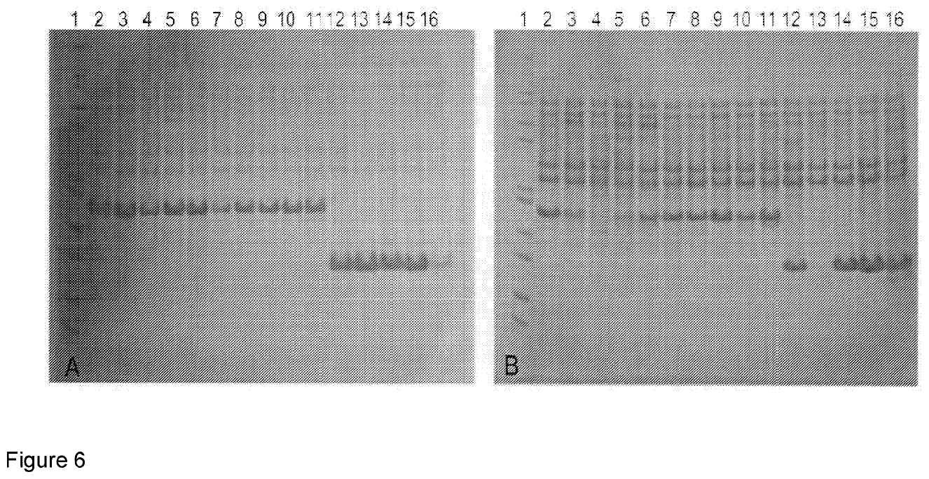

An initial expression test was performed to assess solubility. The results show that all were expressed, but not all were soluble (FIG. 6). Note: solubility (or a lack thereof) is not necessarily a predictor of utility. One of skill will appreciate that when manufacturing or producing proteins, certain processes require the use of insoluble material as this is readily purified (from inclusion bodies and the like). Downstream protocols may then re-engineer proteins to modulate features such as solubility.

Results of the expression test show that:
Im16 (L170A) is insoluble or very poorly soluble
Im25 (TD, S345D) is insoluble
Im15 (Y168W) and Im17 (L170T) have reduced solubility
Im18 (V173G) and Im22 (I286A) are slightly reduced.
The remainder show soluble expression.

The 13 soluble proteins were expressed in E. coli and purified by immobilized metal affinity chromatography (IMAC), followed by TEV digestion to remove the 6His-tag, then reverse IMAC and size exclusion chromatography (SEC).

Ten purified domains (WTSp, Im19, Im20, Im21, Im22, Im23, WTTD, Im24, Im26 and Im27) were further characterized by:
(i) Thermofluor to measure melting temperature (Tm)
(ii) Near UV circular dichroism (CD) to compare tertiary structures to WT
(iii) Dynamic light scattering (DLS) to check oligomeric state in solution
(iv) Surface plasmon resonance (SPR) to measure binding affinity to sialyllactose
(v) Measurement of IL-8 cytokine stimulation

The results are summarized in Table 1.

TABLE 1
Qualitative summary of the biophysical characterizations of the WT domains and their variants. Colour
coding is from green to red (including green′, orange and yellow), where green indicates that the
variant closely resembles its WT counterpart for that particular characteristic and pale green (green′)
or yellow indicate increasing degrees of differences. Red or orange indicate significant differences.

| | | | | Tm +/− | NearUV | | | Cytokine |
|---|---|---|---|---|---|---|---|---|
| Name | Mutation | Solubility | Purification | 6SL | CD | DLS | Biacore | stimulation |
| WTSp | — | Green | Green | Green | Green | Green | Green | Green |
| Im15 | Y168W | Yellow | Orange | Red | N/A | N/A | N/A | N/A |
| Im16 | L170A | Red | N/A | N/A | N/A | N/A | N/A | N/A |
| Im17 | L170T | Yellow | Orange | N/A | N/A | N/A | N/A | N/A |
| Im18 | V173G | Green′ | Orange | N/A | N/A | N/A | N/A | N/A |
| Im19 | V239A | Green | Green | Green | Green | Green | Green | N/D |
| Im20 | V239T | Green | Green | Green′ | Green′ | Green | Green | N/D |
| Im21 | V246G | Green | Green | Green′ | Green | Green | Green | Green |
| Im22 | I286A | Green′ | Green | Green′ | Green | Green | Green | Green |
| Im23 | Y292E | Green | Green | Green′ | Green′ | Green | Yellow | Yellow |
| Im24 | S342D | Green | Green | Green | Green | Green | | |
| Im25 | S345D | Red | N/A | N/A | N/A | N/A | | |
| Im26 | L348D | Green | Green | Green′ | Green | Green | | |
| Im27 | R403K | Green | Green | Green | Green | Green | | |
| WTTD | — | Green | Green | Green | Green | Green | | |

N/A: these characterizations were not performed due to poor solubility/purity of the protein.
N/D: not determined.

### Sp Peptide 167-181:

Im15, Im16, Im17, Im18 are all insoluble or poorly soluble (as stated, this does not necessarily impact on protein utility). These are in the ‘moderately’ antigenic region 167-181 (FYNLFSVSSATKKDE). This region is clearly very sensitive to change.

Earlier results show that M156F, which sits adjacent to L170 (and I286), increases Tm by ˜4° C.

This could therefore be combined with L170T. M156F does not increase predicted immunogenicity.

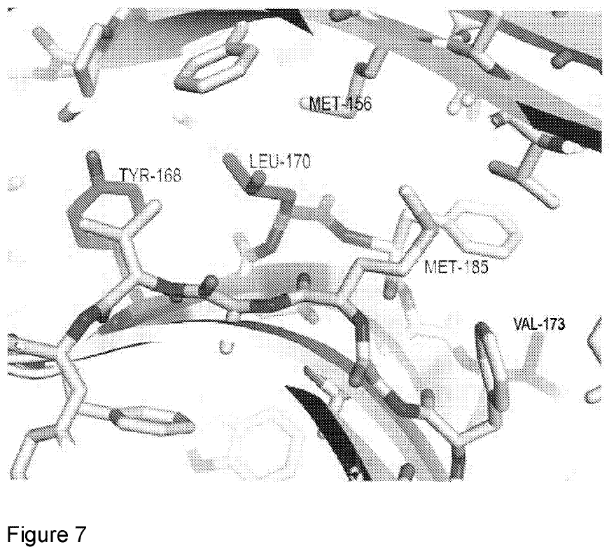

M185I increases Tm by 5° C., and lies parallel to L170 (FIG. 7). This mutation could also be included. Note that, like M156F, M185I does not increase predicted immunogenicity but slightly reduces the number of predicted allele binders.

### Sp Peptide 236-250:

Im19, Im20, Im21 all behave similarly to WT. These are in the ‘highly’ antigenic region 236-250 (KGRVRLYVNGVLSRT).

Im19 (V239A) was chosen over the threonine mutation (Im20, V239T). There is no difference in predicted immunogenicity but Im19 is a closer match to WT Thermofluor Tm and Near UV spectrum. This would be combined with Im21 (V246G).

### Sp Peptide 286-294:

Im22 (I286A) is broadly similar to WT while Im23 (Y292E) appears to exhibit reduced ligand affinity. This region, 286-294 IRNLTVYNR, was not flagged up by ProImmune but is strongly predicted by ProPred to be immunogenic.

There is some indication that Im22 has lower Tm than WT. This residue is adjacent to M156 so may behave differently if M156F was included.

### TD Peptide 338-352:

Im24 (S342D) and Im26 (L348D) show similar characteristics to the WT trimerization domain, but with some suggestion of reduced Tm in Im26. These are in the ‘moderately’ antigenic region 338-352 SDWFSVSSNSLYTLS. The WT sequence was predicted to bind 9 alleles, while Im24 predicts 2 alleles and a Im24/Im26 double mutant predicts 1 allele.

### TD Peptide 392-406:

Im27 (R403K) is similar to WT. It is part of the ‘highly’ antigenic region 392-406 GAQVEVGSLNIRLGT. Predicted alleles are reduced from 21 to 3 when this mutation is introduced.

### Synthesis of Multiple Mutation Combinations Im28-34

The following mutations were introduced:
i) M156F/L170T
ii) M156F/L170T/M185I: In ProPred, alleles predicted for this region are reduced from 31 in the WT to 19 for this combination.
iii) V239A/V246G: In ProPred, alleles for this region are reduced from 44 to 3.
iv) I286A/Y292E: In ProPred, alleles are reduced from 41 to 1.
v) V239A/V246G/I286A/Y292E combines the previous two doubles.
vi) M156F/L170T/M185I/V239A/V246G/I286A/Y292E combines all the Sp mutations
vii) TD: S342D/L348D/R403K: Predicted alleles are reduced from 9 to 1 for TD peptide 338-352 and alleles for peptide TD peptide 392-406 are reduced from 21 to 3. This triple mutant combines all the TD mutants. They are all surface exposed and distal to the N-terminal end of TD, so would not be expected to interfere with SpCBM in the hexamer form.

The constructs are named Im28 to Im34:

| (SpCBM) variant | Mutations |
|---|---|
| Im28 | M156F/L170T |
| Im29 | M156F/L170T/M185I |
| lm30 | V239A/V246G |
| Im31 | I286A/Y292E |
| Im32 | V239A/V246G/I286A/Y292E |
| Im33 | M156F/L170T/M185IA/239A/V246G/I286A/Y292E |
| (PaTD) variant | Mutation |
| Im34 | S342D/L348D/R403K |

### 2.5 Expression and Biophysical Characterization of Im28-Im34

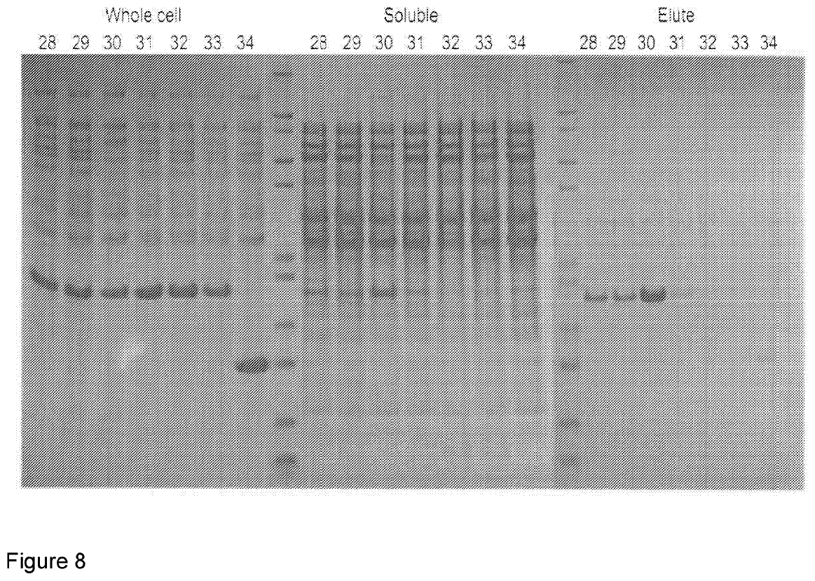

As with the single mutations, the combinations Im28 to Im34 were synthesized by GeneArt and subcloned into pHISTEV for expression analysis. A nickel bead pull-down on the His-tagged soluble extract was also performed (FIG. 8).

### Hexameric Forms

### Design of Hexameric Constructs HEX1 to HEX17

Genes encoding the hexameric forms (called HEX1 to HEX17) were synthesized by GeneArt:

Sp2CBMTD

| variant | Mutations |
|---|---|
| HEX1 | CBM1(L170T V239A V246G I286A Y292E)-CBM2(L170T V239A V246G |
| | I286A Y292E)-TD (S342D L348D R403K) |
| HEX2 | CBM1(V239A V246G I286A Y292E)-CBM2(V239A V246G I286A Y292E)- TD |
| | (S342D R403K) |
| HEX3 | CBM1(V239A V246G I286A)-CBM2(V239A V246G I286A)-TD (S342D |
| | R403K) |
| HEX4 | CBM1(V239A V246G)-CBM2(V239A V246G)-TD(S342D) |
| HEX5 | CBM1(V239A V246G)-CBM2(V239A V246G)-TD(R403K) |
| HEX6 | CBM1(V239A V246G)- CBM2(V239A V246G)-TD(S342D R403K) |
| HEX17 | CBM1(V239A V246G A162P)- CBM2(V239A V246G A162P)-TD (S342D |
| | R403K) |

The hexameric forms were synthesized in two parts to avoid problems associated with synthesising repeat sequences in the tandem CBM copies. The first gene covered the first CBM and the second part encompassed the second CBM plus the TD. These could then be simultaneously cloned into pHISTEV to create the Sp2CBMTD construct that trimerizes upon expression.

The first hexamer, HEX1, contained the mutations L170T/V239A/V246G/I286A/Y292E in the CBMs and S342D/L348D/R403K in the TD.

The solubility data of the individual domains indicated that HEX1 was unlikely to be soluble (again, not necessarily a reflection on the utility of the molecule); a further construct, HEX3, was synthesized. Note that HEX2 contained the same mutations as HEX3, but with the addition of Y292E.

HEX3 was synthesized and subcloned into the pHISTEV vector. Expression was insoluble under all conditions tested (varying temperature, IPTG concentration, cell density at induction, with or without heat shock). The CBM-only domain containing the same three mutations (V239A V246G I286A) is soluble. A double mutant (V239A V246G) behaves very similarly to WT. Therefore, further variants (HEX4, HEX5 and HEX6) were designed and constructed by PCR/ligations, which exclude I286A and contain either one or both of the TD mutations.

During the work on HEX6 a number of other versions were designed containing different combinations of the HEX6 mutations (numbered HEX7 to HEX16; not characterised).

HEX17 contains the HEX6 mutations with an additional A162P mutation. This proline mutation has been shown to increase the single CBM Tm by 3-4° C. The proline mutation is not near the other mutations, the N- or C-termini or the ligand binding site.

### Characterization of the Hexameric Variants

stimulated with the modified hexamer HEX17, IL-8 levels are significantly lower than when compared to Sp2CBMTD (aka SpOrig) stimulated cells.

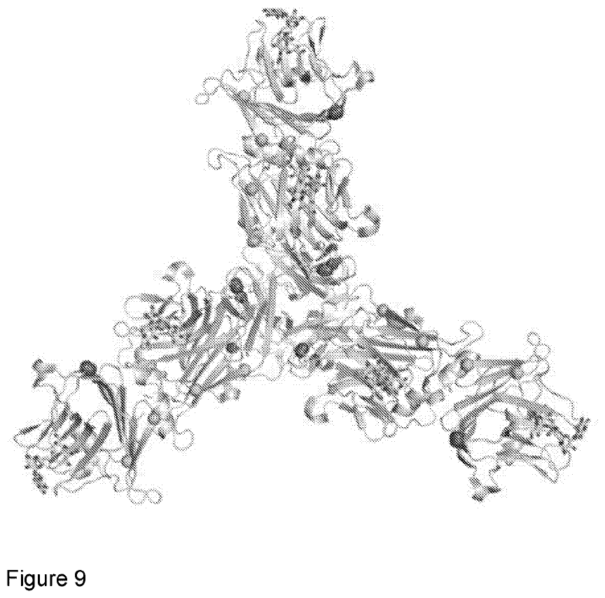

The expression, purification and characterization results are shown in Table 2. Based on these results, HEX6 and HEX17 were taken forward. The positions of the HEX17 mutations on the hexamer are shown in FIG. 9.

TABLE 2
Qualitative summary of the biophysical characterizations of the hexameric Sp2CBMTD variants.
Colour coding is from green to red, where green indicates that the variant closely resembles
its WT counterpart for that particular characteristic and pale green (green′) or yellow
indicate increasing degrees of differences. Red or orange indicate significant differences.

| | | | | | NearUV | | IL-8 |
|---|---|---|---|---|---|---|---|
| Name | Mutations | Solubility | Purification | Thermostability | CD | Biacore | assay |
| Hex1 | L170T/V239A/V246G/I286A/Y292E/ | Red | N/A | N/A | N/A | N/A | N/A |
| | S342D/L348D/R403K | | | | | | |
| Hex2 | V239A/V246G/I286A/Y292E/S342D/ | | | | | | |
| | R403K (designed but not made) | | | | | | |
| Hex3 | V239A/V246G/I286A/S342D/R403K | Red | N/A | N/A | N/A | N/A | N/A |
| Hex4 | V239A/V246G/S342D | Yellow | Red | N/A | N/A | N/A | N/A |
| Hex5 | V239A/V246G/R403K | Green | Yellow | Yellow | N/A | N/A | N/A |
| Hex6 | V239A/V246G/S342D/R403K | Green | Green | Yellow | Green | Green | Yellow |
| Hex7 | Note: These constructs are different combinations of the Hex6 mutations and | | | | | | |
| to 16 | were designed as a back-up in case Hex6 failed | | | | | | |
| Hex17 | A162P/V239A/V246G/S342D/R403K | Green | Green | Green′ | Green | Green | reduced |
| | | | | | | | IL-8 |

N/A: these characterizations were not performed due to poor solubility/purity of the protein.
N/D: not determined.

### Example 3

### Inflammatory Mediators

Aim: To measure the innate immune response of mCBM-treated human lung epithelial cells (A549) by analysing levels of inflammatory mediators over time.

Administration of Sp2CBMTD to mammalian cells stimulated a pro-inflammatory response both in vitro and in vivo^{1,2}. To determine whether this was still observed with modified hexameric sialic acid binding molecules, mammalian A549 cells were stimulated by the addition of 10 μg of biologic (Sp2CBMTD (aka SpOrig), HEX6 (i.e. a sialic acid binding molecule comprising 3×HEX6 units) or HEX17 (i.e. a sialic acid binding molecule comprising 3×HEX17 units) and cell culture medium was harvested at specific time-points post administration. The concentrations of inflammatory mediators were measured both by ELISA and a multiplex assay.

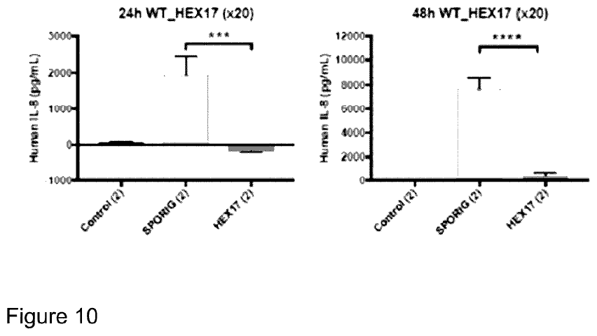

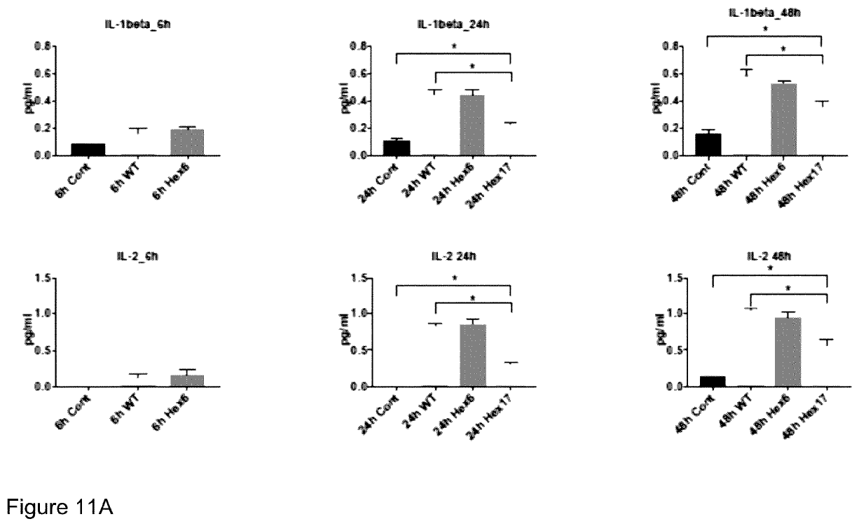

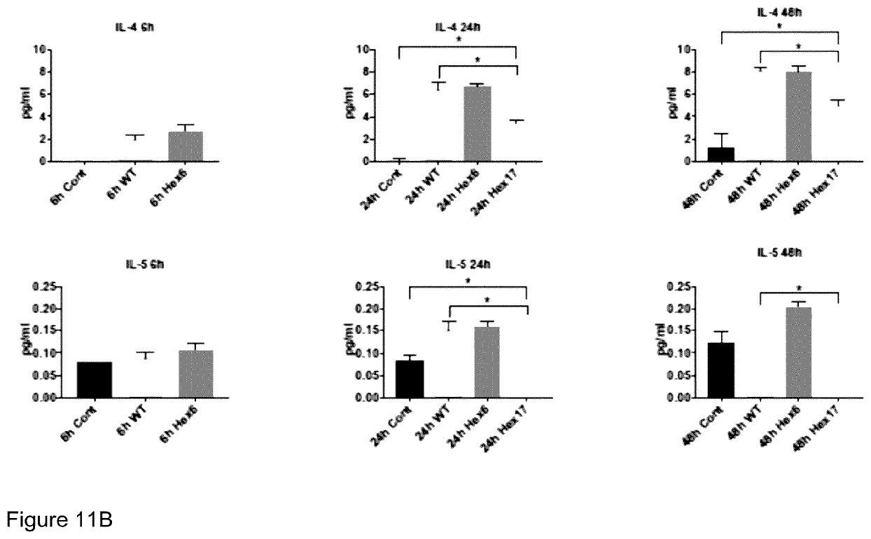

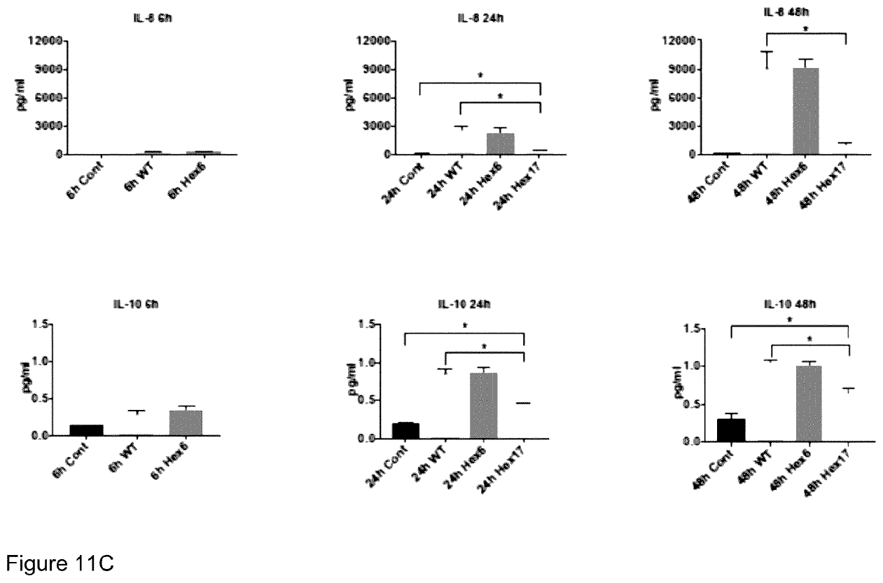

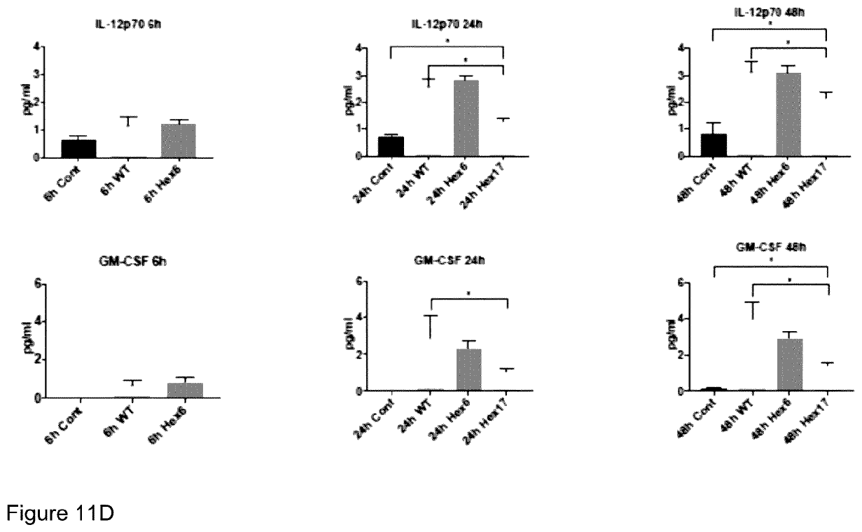

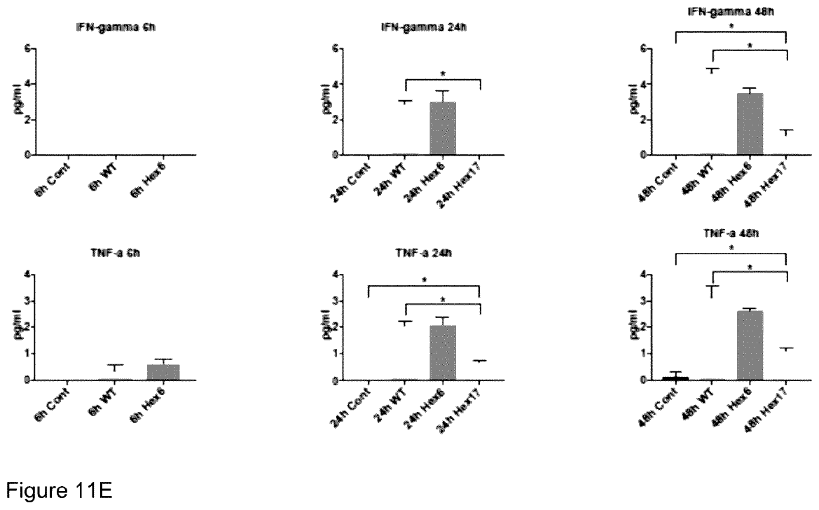

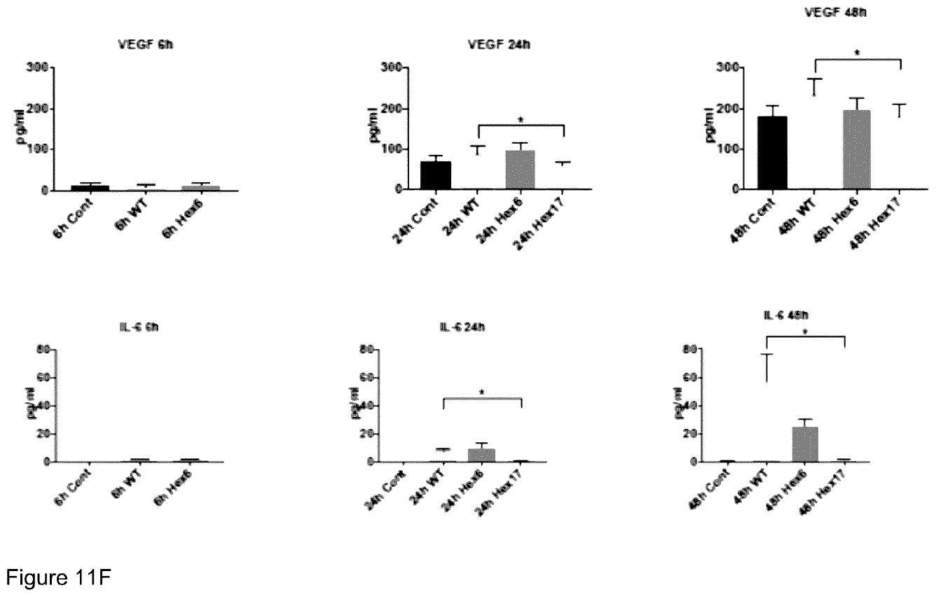

Human IL-8 (benchmark cytokine for the study) response using a human 1×Mouse CXCL1/KC Quantikine ELISA Kit (R&D BioSystems). The concentration levels of IL-8 from stimulated A549 cells are shown in FIG. 10. It is evident that when A549 cells are Inflammatory mediator response using a Human Cytokine 12-plex Assay (Bio-Plex Pro™, Bio-Rad). FIGS. 11A-11F demonstrate the analysis of 12 inflammatory mediators from culture medium after A549 cell stimulation by Sp2CBMTD (WT, aka SpOrig), HEX6 and HEX17 (variants) at specific time points (6 h, 24 h, 48 h). Prior to analysis, samples were thawed and diluted 1:4 in PBS before using a human HS Cytokine-12 plex assay (R&D Systems). The data indicates that:
HEX17 affects the levels of almost all the cytokines tested compared to SpOrig and HEX6. There is a significant reduction in observed concentration (pg/ml) with analytes IL-6, IL-8, GM-CSF and IFN-gamma at 48 h when compared to SpOrig and HEX6.
When compared to control at 48 h, HEX17 appears to cause an increase in the level of all cytokines tested with the exception of IL-5, and VEGF (yet to be confirmed).
HEX6 only showed reduced IL-6 stimulation compared to SpOrig at 48 h.

### Example 4

### In Vivo PR8 Mouse Data

The objective of the study was to assess the efficacy of Sp2CBMTD (SpOrig) and its variants, in a mouse model of lethal influenza infection. Each of the candidate proteins were also administered in the absence of an influenza infection to assess whether they alone, caused any morbidity or mortality.

### Survival, Clinical Scores and Weight Loss.

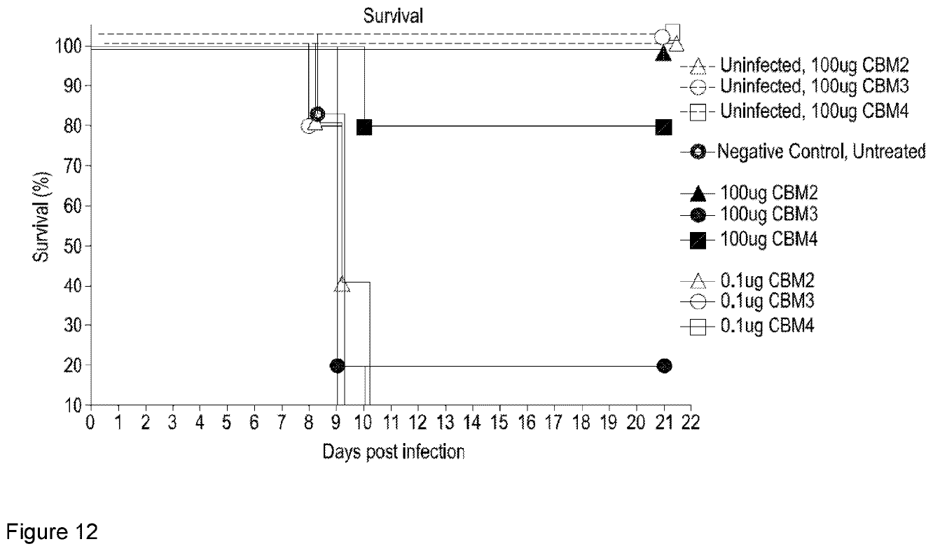

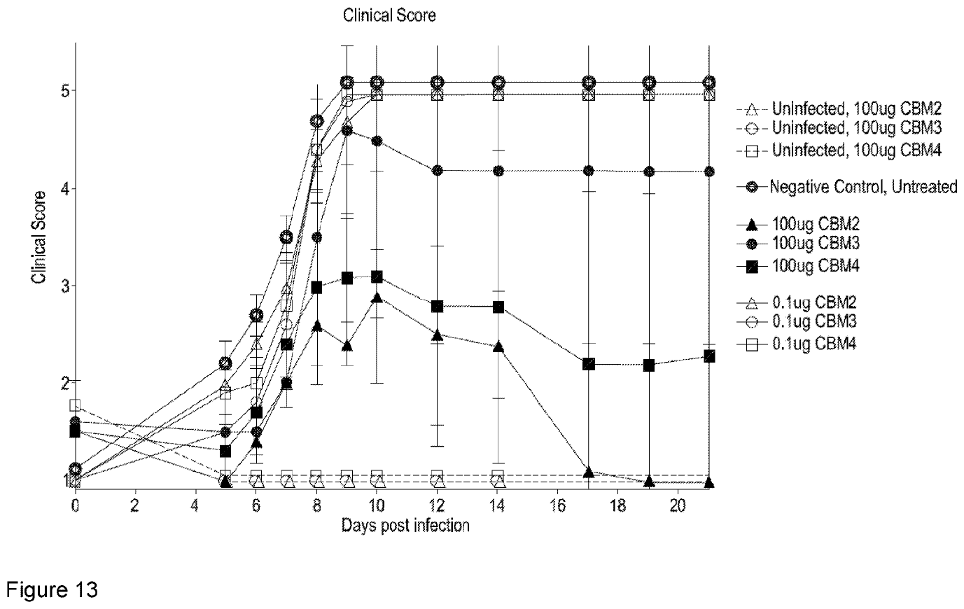

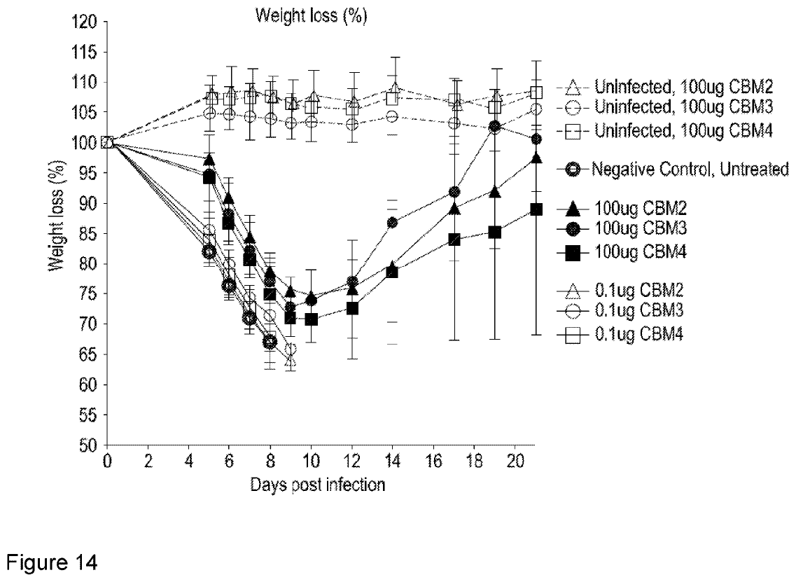

The results show that none of CBM2 (HEX17), CBM3 (HEX6) or CBM4 (WT, SpOrig) caused any overt morbidity or mortality alone. Administration of a single 100 μg dose of either CBM2 (HEX17), CBM3 (HEX6) and CBM4 (WT, SpOrig) one day prior to a lethal challenge with PR8 influenza virus elicited protection against PR8 infection, with greatest efficacy seen with HEX17 (100% survival), followed by SpOrig and then HEX6 (FIG. 12). Clinical scores were also lower with HEX17 compared to SpOrig and HEX6 (FIG. 13). Mice from single high dose treated groups that survived all lost weight at peak infection but soon recovered, in contrast to untreated, infected mice (FIG. 14).

Anti-mCBM Antibody Analysis of Lung Homogenates and Sera Tissue from a PR8-Challenged Mouse Study.

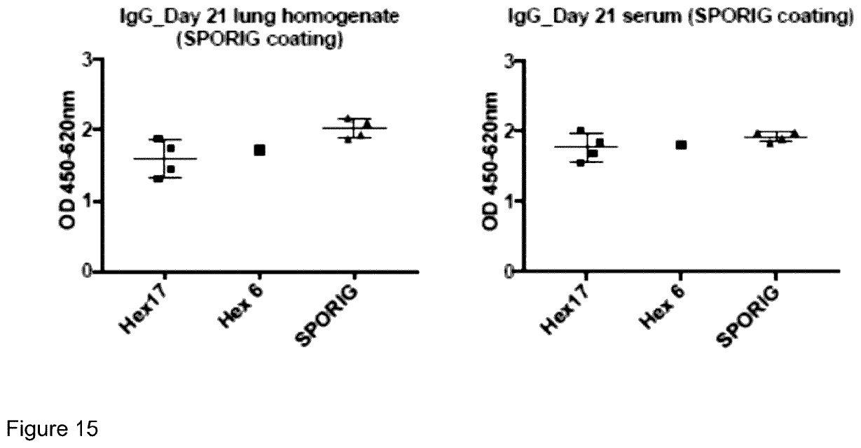

The objective of this study was to determine whether modified immunogenic epitopes of the modified variants of Sp2CBMTD (SpOrig) demonstrated reduced antibody levels in mice in a PR8-challenged study (it should be noted that epitopes were modified based on human MHC-class II binding information). For this, survived mice from PR8-challenged study were culled at Day 21 with lung and sera harvested and tested for anti-mCBM antibodies—IgG, IgA, IgE and IgM against coated antigen SpOrig (1 μg/well) in an ELISA format. The data shown in FIG. 15 indicated that:
The modified protein HEX17 did show a significant (p<0.05) reduction in mouse lung IgM levels compared to SPORIG. Due to only one surviving mouse for HEX6 treatment, only HEX17 and SPORIG data was statistically analysed.
There is some indication of a slight downward trend of antibody levels in mice (lung IgA and IgM) that were treated with the modified CBMs compared to SpOrig.

### Example 5

The aim of the following experiments is to determine whether there is a direct interaction between CBMs (Sp2CBMTD and Vc2CBMTD) and human RSV in a cell-free system. ELISA experiments were designed as follows:

### Experiment 1

96-well plate was pre-coated with different concentrations of RSV (strain type A2), CBMs, Sp2CBMTD and Vc2CBMTD and PBS (vehicle, negative control) overnight at 4° C. Wells were blocked with Blocking Buffer (BB, PBS containing 1% w/v BSA). Different concentrations of CBMs were diluted in BB and then added to the wells after removing coating antigens and blotting the plate. The plate was left to incubate for 1 hr at room temperature. Wells were then washed with Wash Buffer (WB, PBS supplemented with 0.5% (v/v) Tween-20) before adding primary antibodies (rabbit anti-SpCBM antibody or rabbit anti- VcCBM antibody, 1:5000 dilution in BB, Eurogentec) and left to incubate for 1-2 hr at room temperature. Goat anti-rabbit IgG-HRP (1:2500 dilution in BB, Sigma) was used as the secondary antibody and TMB (Sigma) was used for substrate. CBM binding to RSV was determined by comparing the absorbance at 450 nm (620 nm as background reference) of treated versus untreated control wells after reactivity with TMB (substrate for HRP).

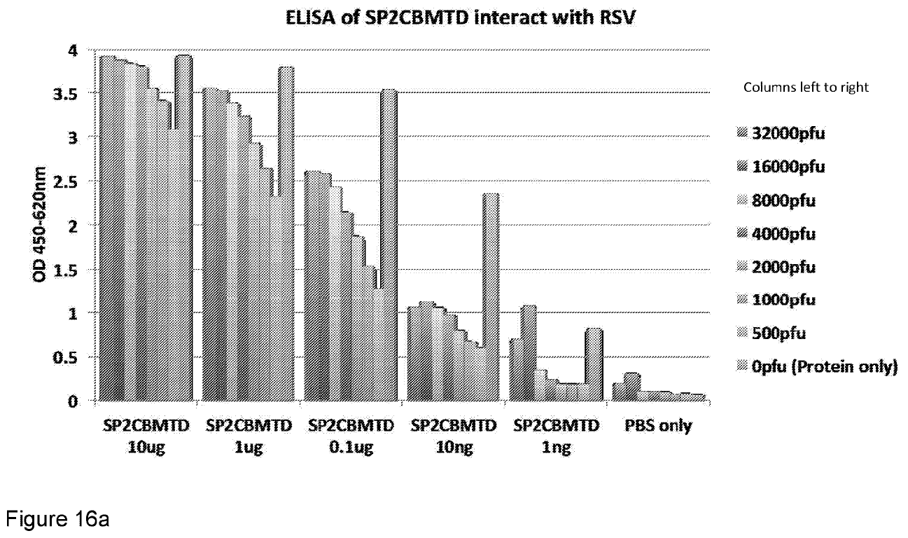

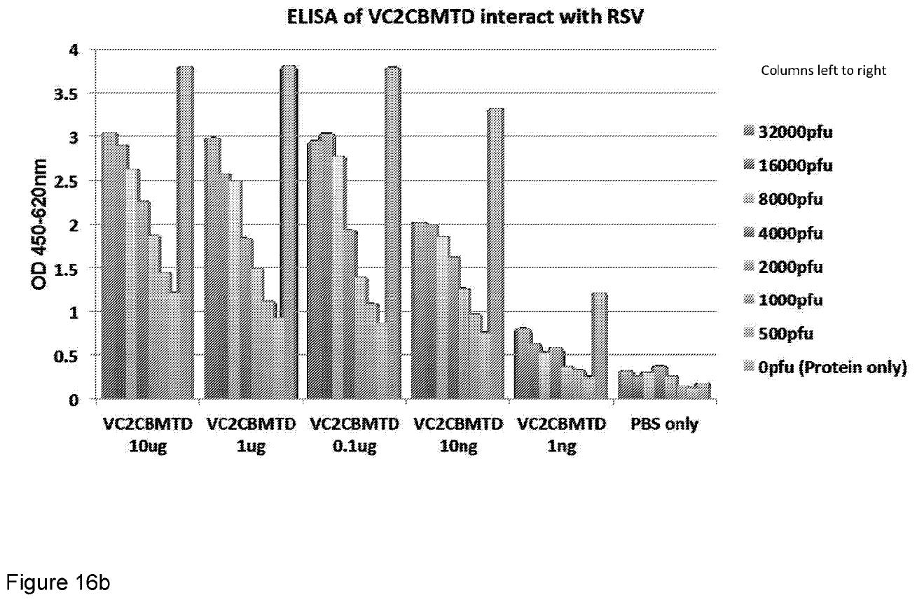

The results shown both Sp2CBMTD and Vc2CBMTD interact directly with RSV in a dose-dependent manner (see FIGS. 16A and B).

### Experiment 2

A 96-well plate was pre-coated with 10 μg of either GFP-Sp2CBMTD, GFP-Vc2CBMTD, GFP, GFP-Sp2CBMTD-R274Q (a sialic acid binding mutant of Sp2CBMTD) or 32000 PFU RSV as coating antigens and PBS (vehicle, negative control). Wells were incubated with relevant proteins for 1 hr at room temperature. Images of GFP fluorescent binding were captured using a microscope (in the GFP channel on EVOS FL Cellular Imaging System) with 10× objective.

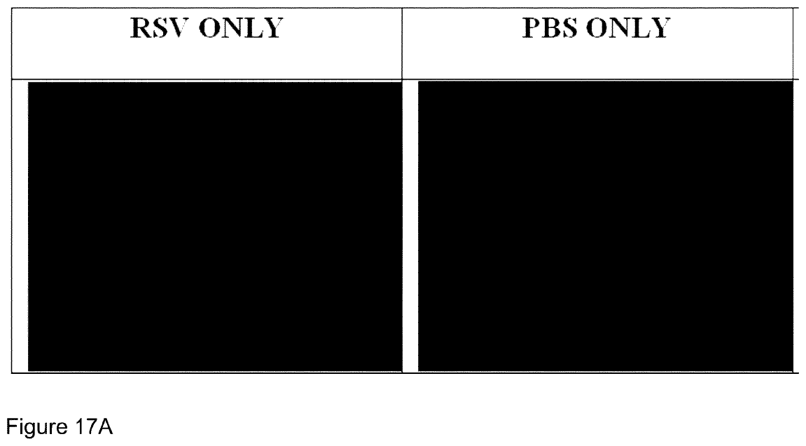

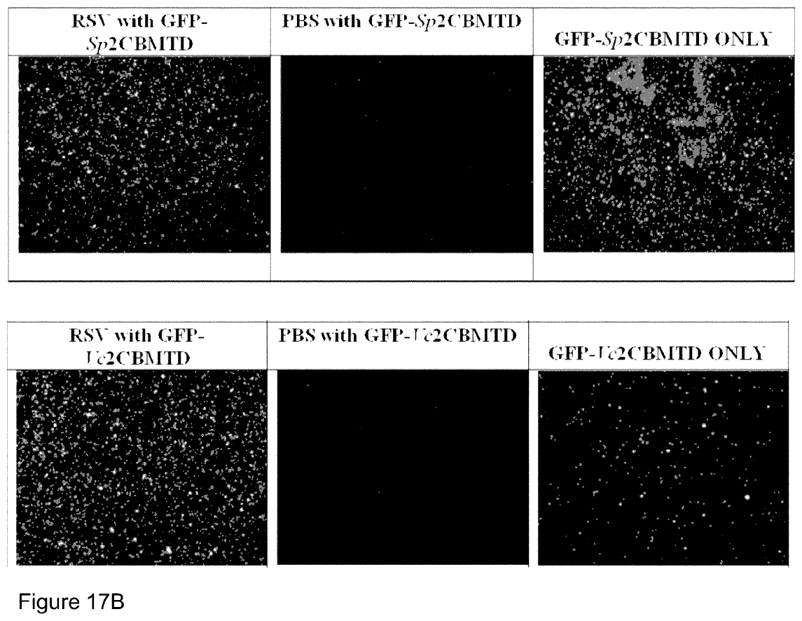

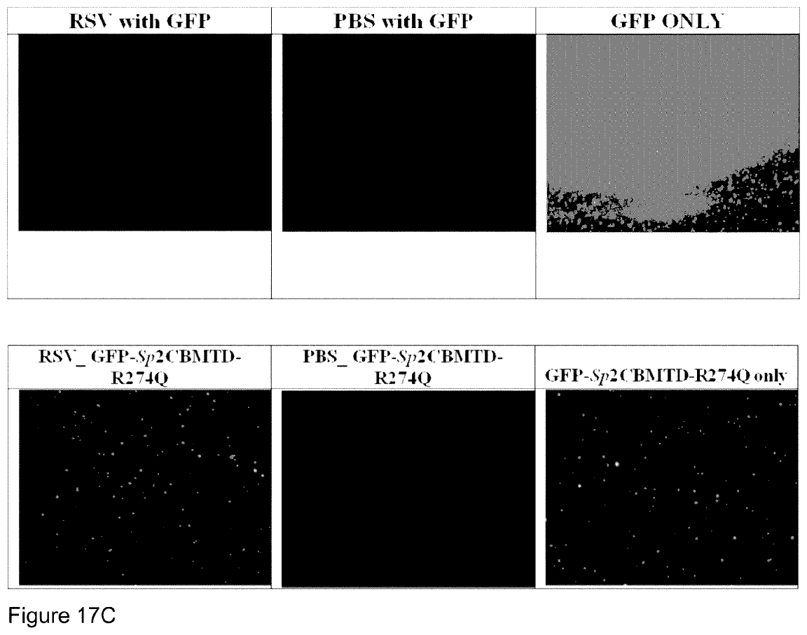

The results indicate that both GFP-Sp2CBMTD and GFP-Vc2CBMTD are able to bind directly to RSV. Negligible binding to RSV was observed with the sialic acid binding mutant GFP-Sp2CBMTD-R274Q (see FIGS. 17A-E)

### Example 6

The aim of the following experiments is to support the observation that the interactions between CBMs (Sp2CBMTD and Vc2CBMTD) and RSV are based on CBMs binding to the viral surface glycoproteins terminating with sialic acid. Thus, three ELISA experiments were designed.

### Experiment 1

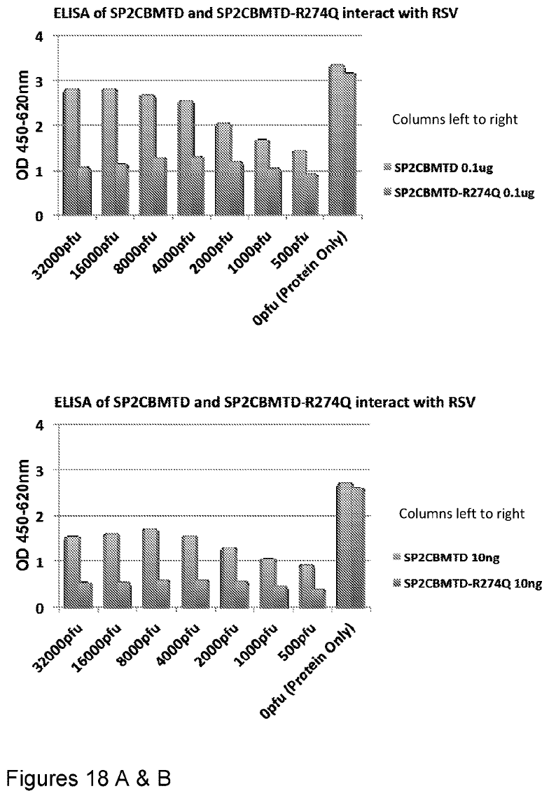

Both wild type Sp2CBMTD and mutant Sp2CBMTD-R274Q (sialic acid binding mutant) were used in an ELISA format as described in Experiment 1, Example 5. The results demonstrate that wild type Sp2CBMTD binds with much higher affinity to RSV than compared with the same concentration of mutant Sp2CBMTD-R274Q (see FIGS. 18A and B).

### Experiment 2

Neuraminidase (Nu) from Clostridium perfringens (Sigma) is a glycosidase that is able to cleave terminal sialic acids from cell surface sialylated glycoconjugates, with linkage specificity for α-2,3- α-2,6- α-2,8-linked to Gal, GlcNac, GalNAc, AcNeu, GlcNeu, oligosaccharides, glycolipids or glycoproteins. To test whether this is the case for viral surface glycoproteins, RSV was treated with and without neuraminidase prior to the addition of CBMs. An ELISA experiment similar to Experiment 1 in Example 5, was set up. A 96-well plate was pre-coated with different concentration of RSV, CBMs (as positive controls) and PBS (as negative control). For some RSV-coated wells, neuraminidase (30 mU/well) was added for 1 hr followed by adding CBMs (100 ng or 10 ng). CBM binding was evaluated using anti-CBM antibodies followed by a detection anti-IgG-HRP as described in Project 1.

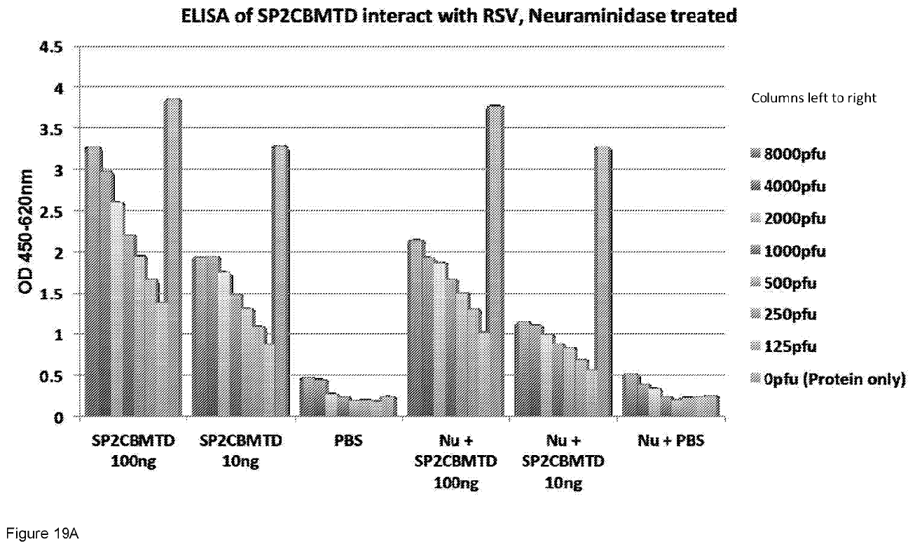

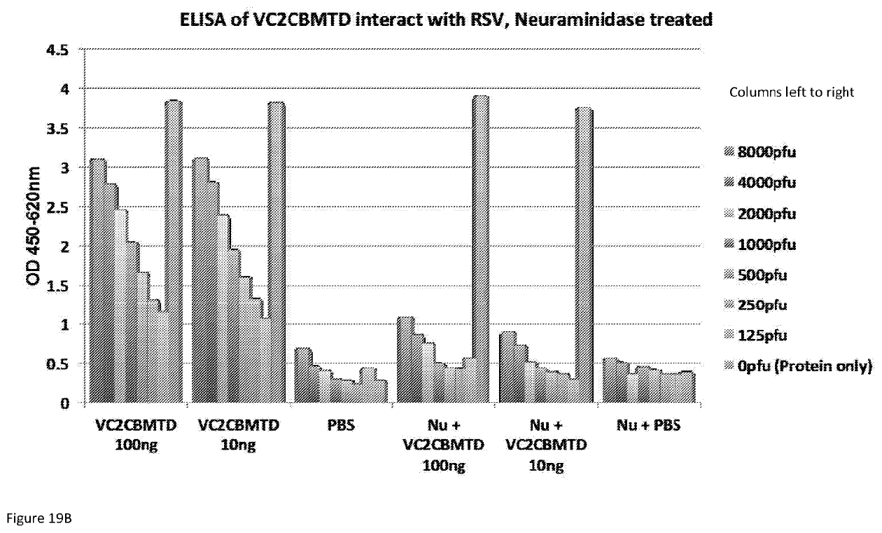

The results demonstrate that in the absence of neuraminidase treatment, both Sp2CBMTD and Vc2CBMTD interact with RSV in a dose-dependent manner as observed previously (below, and Experiment 1, Example 5). The interaction is partially abolished after neuraminidase treatment of RSV in the presence of Sp2CBMTD, but almost completely abolished when using Vc2CBMTD as the interacting partner (see FIGS. 19A and B).

This data provides evidence that RSV surface glycoproteins are sialylated suggesting a potential directly acting antiviral role of CBMs in RSV infection.

### Experiment 3:

To determine whether a competing ligand can block the sialic acid binding site of CBMs and interfere with the interaction of CBM with RSV, CBMs (Sp2CBMTD and Vc2CBMTD, 100 ng/well) were pre-incubated with different concentrations of α2,3 sialyllactose (3′-SL, 10 mM, 100 mM, Glycotech) for 1 hr at room temperature. The mixture was added to a 96-well plate coated with the RSV (32000 PFU/well) along with controls (PBS, CBM only). CBMs were added to wells as in the ELISA experiment as in Example 5. The number of replicates in the study was n=3, Raw data were analyzed by one-way ANOVA using GraphPad Prism. A p value <0.05 was considered statistically significant (*p<0.05; **p<0.01; ***p<0.001; ****p<0.0001).

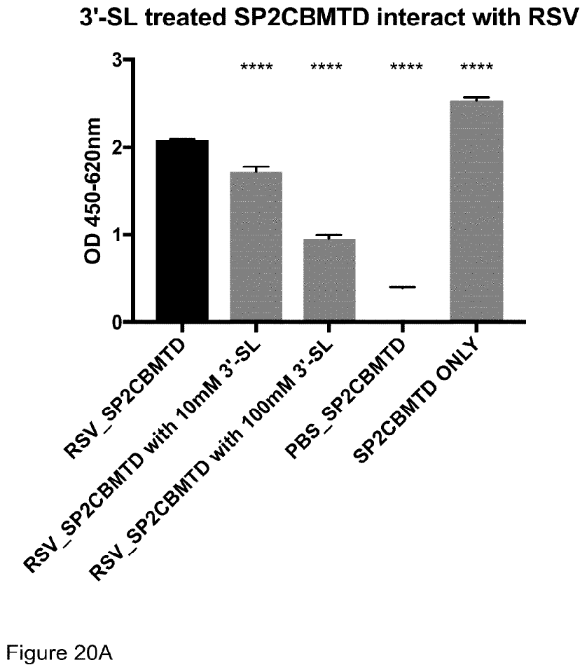

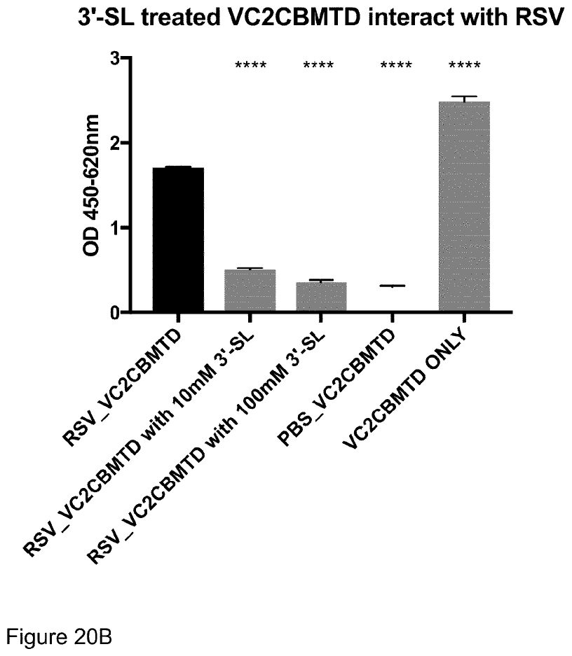

The result shown that a competing sialoside, 3′-SL is able to reduce the interaction between RSV and Sp2CBMTD and Vc2CBMTD in a dose-dependent manner (see FIGS. 20A and B).

### Example 7

An in-vitro study was undertaken to prove that mammalian HEp2:NPro cell protection by CBMs (Sp2CBMTD and Vc2CBMTD) from RSV is decreased after cells are treated using neuraminidase.

The experiment was designed by incubating attached HEp2:NPro cells in a 96-well plate with or without neuraminidase for 1 hr at 37° C., 5% CO₂, followed by CBMs (100 μg/well) for a further 1 hr, and finally, the addition of RSV (3000 PFU/well) for 1 hr. The plate was then overlaid with AVICEL® (Sigma) medium and left to incubate for 96 hr at 37° C., 5% CO₂. RSV-infected cells were determined by evaluating the quantity of RSV that infected the cells using a mouse anti-RSV F antibody (1:200 dilution, MCA490, BioRad) followed by a secondary anti-mouse IgG-HRP antibody (1:2000 dilution, Cell Signalling Technology). Inhibition of RSV infectivity was determined by comparing the absorbance at 450 nm (620 nm as background reference) of neuraminidase-treated versus untreated control wells after reactivity with TMB (substrate for HRP). Replicates n=4, Raw data were analyzed by one-way ANOVA using GraphPad Prism. A p value <0.05 was considered statistically significant (*p<0.05; **p<0.01; ***p<001; ****p<0.0001).

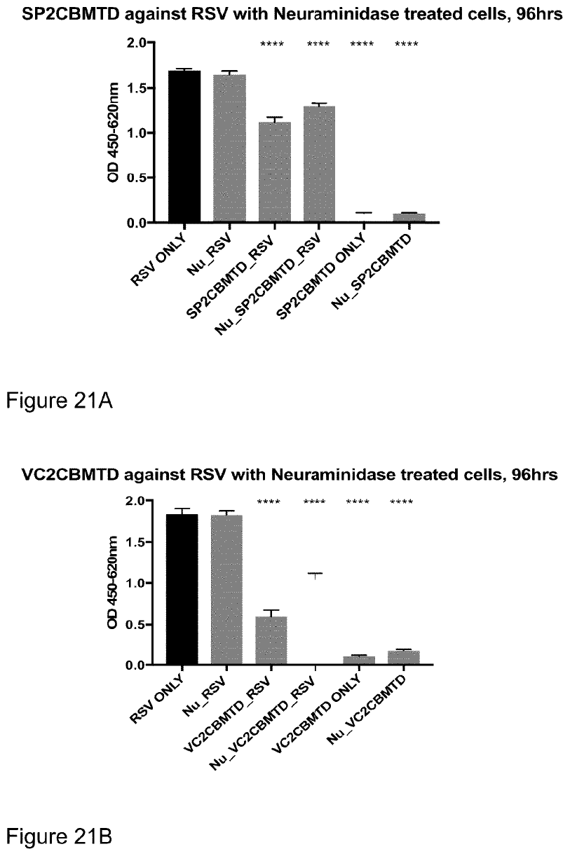

For RSV only control, the result does not show any difference in infectivity between neuraminidase-treated and untreated cells (first two bars on graphs). However, cell protection from both CBMs is decreased after neuraminidase treatment of cells (fourth bar on graphs) but not to the same extent as cells being initially treated by CBMs followed by RSV, this being more evident with Vc2CBMTD. The data suggests that CBMs also protect cells against RSV by targeting cell surface sialic acid (See FIGS. 21A and B).

### Example 8

Utility of engineered carbohydrate-binding module proteins (Sp2CBMTD and Vc2CBMTD) on RSV-B infection using an in vitro 3D model of the human respiratory epithelium.

The objective of this study is to evaluate the effect of mCBM40s, Sp2CBMTD and Vc2CBMTD upon RSV replication in a 3D model of the human respiratory epithelium. The assay was performed using MucilAir™, which is a fully differentiated and ready-to-use 3D model of human airway epithelium, constituted with primary human epithelial cells freshly isolated from nasal, tracheal or bronchial biopsies (Epithelix). For infection studies, the RSV-B strain (10⁶ genome copies/ml RSV, Geneva clinical strain, 2017) was used and applied to the apical side of pre-washed (with PBS) MucilAir inserts on Day 0. For mCBM treatments (n=3), Sp2CBMTD and Vc2CBMTD (at 10 μg or at 100 μg doses) were applied apically either as a single dose one hour before viral infection, or b) as a repeated dose one day and one hour before viral infection. Both regimens had a further exposure of mCBM at 3.5 h post-infection (p.i). Untreated, infected MucilAir (n=2) was used as a control. Apical washes (200 μL) were collected at 3.5 h p.i. and then at 24, 48 and 96 h p.i.

Viral genome copy number was determined using 20 μL of apical wash for viral RNA extraction using the QIAampR Viral RNA kit (Qiagen). Viral RNA was quantified using QuantiTect Probe RT-PCR (Qiagen) using 5 μL of viral RNA and two RSV-B specific primers. Data from readout was corrected for dilution factor and presented as genome copy number per ml. Statistical analysis was performed using Prism 6.0 GraphPad software. Student's unpaired t test was used to compare two sets of data, where *p<0.05.

1. A method for treating and/or preventing symptoms, infections, diseases and/or conditions associated with respiratory syncytial virus (RSV), said method comprising administering a sialic binding molecule to a subject in need thereof.

2. A method of neutralising or blocking a RSV infection, said method comprising administering a sialic binding molecule to a subject in need thereof.

3. An in vitro method of rendering cells non-permissive to RSV, said method comprising contacting or incubating cells susceptible or vulnerable to RSV infection with a sialic acid binding molecule under conditions which permit binding between the sialic acid binding molecule and cell surface sialic acid/sialoglycoconjugates.

4. The method of claim 1, said method comprising mucosally administering a composition comprising the sialic binding molecule to the subject in need thereof.

5. The method of claim 1, wherein the sialic acid binding molecule is administered prophylactically to prevent a RSV infection.

6. The method of claim 1, wherein the sialic acid binding molecule does not exhibit sialidase activity.

7. The method of claim 1, wherein the sialic acid binding molecule does not bind heparin or heparin sulfate and/or comprise the GAG-binding domain of a protein that binds heparin or heparin sulfate.

8. The method of claim 1, wherein the sialic acid binding molecule comprises one or more carbohydrate binding module(s).

9. The method of claim 1, wherein the sialic acid binding molecule comprises one or more family 40 carbohydrate binding module(s).

10. The method of claim 1, wherein the sialic acid binding molecule comprises the sialic acid binding domain of Vibrio cholerae NanH sialidase and/or the sialic acid binding domain of Streptococcus pneumoniae NanA sialidase.

11. The method of claim 10, wherein the Vibrio cholerae NanH sialidase comprises the amino acid sequence of SEQ ID NO: 1 or 2.

12. The method of claim 10, wherein the Streptococcus pneumoniae NanA sialidase comprises the amino acid sequence of SEQ ID NO: 3 or 4.

13. The method of claim 1, wherein the sialic acid binding molecule is Sp2CBM, Sp2CBMTD, Vc4CBM, Vc2CBM or Vc2CBMTD.

14. The method of claim 1, wherein the sialic acid binding molecule comprises one or more modified Family 40 carbohydrate binding modules (CBM40(s)).

15. The method of claim 14, wherein the one or more modified CBM40(s) contain(s) one or more mutations relative to a reference sequence and wherein the reference sequence is selected from the group consisting of:
(i) a wild type Family 40 CBM sequence;
(ii) a wild type CBM40 sequences from Vibrio cholerae;
(iii) the NanH sialidase sequence of Vibrio cholerae;
(iv) a wild type CBM40 sequences from Streptococcus pneumoniae;
(v) the NanA sialidase sequence of Streptococcus pneumoniae;
(vi) the sequence of SEQ ID NO: 1;
(vii) the sequence of SEQ ID NO: 2;
(viii) the sequence of SEQ ID NO: 3; and
(ix) the sequence of SEQ ID NO: 4.

16. The method of claim 15, wherein the mutation is selected from the group consisting of:
(i) one or more amino acid substitution(s);
(ii) one or more amino acid deletion(s);
(iii) one or more amino acid addition(s)/insertion(s);
(iv) one or more amino acid/sequence inversions; and
(v) one or more amino acid/sequence duplications.

17. The method of claim 14, wherein the sialic acid binding molecule comprises a modified oligomerisation domain.

18. The method of claim 17 wherein the modified oligomerisation domain contains one or more mutations relative to a reference sequence and wherein the reference sequence is selected from the group consisting of:
(i) a wild type Pseudomonas aeruginosa pseudaminidase sequence;
(ii) the Pseudomonas aeruginosa pseudaminidase amino acid sequence deposited under accession number Q9L6G4;
(iii) the sequence of SEQ ID NO: 5; and
(iv) the sequence of SEQ ID NO: 6.

19. The method of claim 1, wherein the sialic acid binding molecule comprises the following structure:
CBM1(V239A V246G A162P)--CBM2(V239A V246G A162P)--TD(S342D R403K) wherein CBM1 and CBM 2 are derived from CBM40 sequences and TD is derived from a trimerisation domain.

20. The method of claim 1, wherein the sialic acid binding molecule comprises the following sequence:
GAMVIEKEDVETNASNGQRVDLSSELDKLKKLENATVHMEFKPDPKAPAF
YNLFSVSSATKKDEYFTMAVYNNTATLEGRGSDGKQFYNNYNDAPLKVKP
GQWNSVTFTVEKPTAELPKGRARLYVNGGLSRTSLRSGNFIKDMPDVTHV
QIGATKRANNTVWGSNLQIRNLTVYNRALTPEEVQKRSGGGSGVIEKEDV
ETNASNGQRVDLSSELDKLKKLENATVHMEFKPDPKAPAFYNLFSVSSAT
KKDEYFTMAVYNNTATLEGRGSDGKQFYNNYNDAPLKVKPGQWNSVTFTV
EKPTAELPKGRARLYVNGGLSRTSLRSGNFIKDMPDVTHVQIGATKRANN
TVWGSNLQIRNLTVYNRALTPEEVQKRSGGSLGVPDFESDWFDVSSNSLY
TLSHGLQRSPRRVVVEFARSSSPSTWNIVMPSYFNDGGHKGSGAQVEVGS
LNIKLGTGAAVWGTGYFGGIDNSATTRFATGYYRVRAWI
